(19) United States
(12) Patent Application Publication
(10) Pub. No.: US 2009/0005444 A1
(43) Pub. Date: Jan. 1, 2009

(54) USE OF PROPOFOL PRODRUGS FOR TREATING ALCOHOL WITHDRAWAL, CENTRAL PAIN, ANXIETY OR PRURITUS

(75) Inventors: Kenneth C. Cundy, Redwood City, CA (US); Peter A. Virsik, Portola Valley, CA (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(73) Assignee: XenoPort, Inc., Santa Clara, CA (US)

(21) Appl. No.: 12/141,636

(22) Filed: Jun. 18, 2008

Related U.S. Application Data
(60) Provisional application No. 60/945,541, filed on Jun. 21, 2007.

(51) Int. Cl. A61K 31/265; A61P 25/00

(52) U.S. Cl. 514/512

(57) ABSTRACT

Methods of treating alcohol withdrawal, central pain, anxiety, or pruritus in a patient comprising orally administering a therapeutically effective amount of a propofol prodrug having high oral bioavailability are disclosed.

This application claims the benefit under 35 U.S.C. §119(e) of U.S. Provisional Application Ser. No. 60/945,541, filed Jun. 21, 2007, which is incorporated by reference herein in its entirety.

### FIELD

Disclosed herein are methods of treating alcohol withdrawal, central pain, anxiety, or pruritus using propofol prodrugs having high oral bioavailability.

### BACKGROUND

Propofol (2,6-diisopropylphenol),

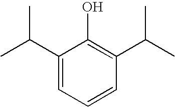

is a low molecular weight phenol that is widely used as an intravenous sedative-hypnotic agent in the induction and maintenance of anesthesia and/or sedation in mammals. The advantages of propofol as an anesthetic include rapid onset of anesthesia, rapid clearance, and minimal side effects (Langley et al., Drugs 1988, 35, 334-372). The hypnotic effects of propofol may be mediated through interaction with the GABA_{A} receptor complex, a hetero-oligomeric ligand-gated chloride ion channel (Peduto et al., Anesthesiology 1991, 75, 1000-1009).

Propofol also has a broad range of other biological and medical applications, which are evident at sub-anesthetic (e.g., sub-hypnotic) and sub-sedative doses. There is evidence that propofol can be effective in treating alcohol withdrawal, and more specifically delirium tremens (see, e.g., DeBellis et al., J. Intensive Care Med 2005, 20(3), 164-73; McCowan and Marik, Crit Care Med 2000, 28(6), 1781-4; Coomes and Smith, Ann Emerg Med 1997, 30, 825-828; Takeshita, J Clin Psychiatry 2004, 65(1), 134-5; and Stiebel et al., Psychosomatics 1994, 35, 193). Propofol has been shown to be effective in the treatment of central pain such as trigeminal neuralgia (Kubota et al., Exp Brain Res. 2007, 179(2), 181-190), spinal cord injury (SCI) pain (Canavero and Bonicalzi, Neurol Sci 2001, 22, 271-273; and Canavero and Bonicalzi, Clin Neuropharmacol 2004, 27(4), 182-186), and central post-stroke pain (CPSP) (Canavero et al., J Neurol 1995, 242(9), 561-567; and Canavero and Bonicalzi, Pain 1998, 74(2-3), 109-114). Propofol has been shown to act as an anxiolytic, especially in the surgical setting (Rothermel, Curr Opin Pediatr 2003, 15(2), 200-203; and Bal et al., Eur J Anaesthesiol 2006, 23(6), 470-475) and preclinical studies demonstrate that the anxiolytic effect is independent of any sedative effect (Pain et al., Anesthesiology 1999, 90(1), 191-196; and Kurt et al., Pol J Pharmacol 2003, 55, 973-977). At subhypnotic doses, propofol has also been shown to be effective in controlling pruritus associated with cholestasis and spinal morphine administration (Borgeat et al., Anesthesiology 1992, 76, 510-512; Borgeat et al., Am J Gastroenterol 1992, 87, 672-674; Borgeat et al., Gastroenterology 1993, 104, 244-247; Bergasa, J Hepatology 2005, 43, 1078-1088; Horta et al., Br J Anaesthesia 2006, 96(6), 796-800; and Kostopanagiotou et al., Eur J Anaesthesiol 2006, 23(5), 418-21).

Propofol is rapidly metabolized in mammals with the drug being eliminated predominantly as glucuronidated and sulfated conjugates of propofol and 4-hydroxypropofol (Langley et al., Drugs 1988, 35, 334-372). Propofol is poorly absorbed in the gastrointestinal tract and only from the small intestine. When orally administered as a homogeneous liquid suspension, propofol exhibits an oral bioavailability of less than 5% that of an equivalent intravenous dose of propofol. Propofol clearance exceeds liver blood flow, which indicates that extrahepatic tissues contribute to the overall metabolism of the drug. Human intestinal mucosa glucuronidates propofol in vitro and oral dosing studies in rats indicate that approximately 90% of the administered drug undergoes first pass metabolism, with extraction by the intestinal mucosa accounting for the bulk of this pre-systemic elimination (Raoof et al., Pharm. Res. 1996, 13, 891-895). Because of its poor oral bioavailability and extensive first-pass metabolism, propofol is administered by injection or intravenous infusion and oral administration of propofol has not been considered therapeutically effective. This has prevented investigations into the efficacy of propofol for treating chronic pathologies and diseases or conditions for which intravenous infusion is not appropriate.

Recently, several methods for improving propofol absorption from the gastrointestinal tract and/or minimizing first pass metabolism have been demonstrated. For example, propofol prodrugs that exhibit enhanced oral bioavailability and that are sufficiently labile under physiological conditions to provide therapeutically effective concentrations of propofol following oral administration have been described by Gallop et al., U.S. Application Publication Nos. 2005/0004381, 2005/0107385, and 2006/0287525; and Xu et al., U.S. Application Publication Nos. 2006/0041011, 2006/0100160, and 2006/0205969, each of which is incorporated by reference herein in its entirety. These propofol prodrugs provide improved oral bioavailability of propofol and can also facilitate oral propofol regimens capable of providing therapeutically effective blood concentrations of propofol appropriate for treating chronic diseases and disorders. Propofol prodrugs that provide a high oral bioavailability of propofol, such as the propofol prodrugs disclosed by Gallop et al. and by Xu et al., enable the use of propofol for treating diseases where it is desirable to administer propofol orally.

### SUMMARY

Accordingly, methods of treating alcohol withdrawal, central pain, anxiety, or pruritus in a patient are disclosed comprising orally administering to a patient in need of such treatment a therapeutically effective amount of a propofol prodrug that is capable of providing a high oral bioavailability of propofol. These and other features of the present disclosure are set forth herein.

### BRIEF DESCRIPTION OF THE DRAWINGS

The skilled artisan will understand that the drawings, described herein, are for illustration purposes only. The drawings are not intended to limit the scope of the present disclosure.

FIG. 1 shows propofol blood concentrations following oral administration of compound (2) to rats at doses from 25 mg-equivalent/kg to 300 mg-equivalent/kg of propofol.

FIG. 2 shows propofol blood concentrations following oral administration of compound (2) to rats at doses from 400 mg-equivalent/kg to 800 mg-equivalent/kg of propofol.

FIG. 3 shows propofol blood concentrations following oral administration of compound (2) to dogs at doses from 50 mg-equivalent/kg to 150 mg-equivalent/kg of propofol.

### DETAILED DESCRIPTION

### Definitions

A dash (“-”) that is not between two letters or symbols is used to indicate a point of attachment for a substituent. For example, —CONH₂ is attached through the carbon atom.

“Alkyl” by itself or as part of another substituent refers to a saturated or unsaturated, branched, or straight-chain monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, an alkyl group can have from 1 to 20 carbon atoms (C₁₋₂₀) in certain embodiments, from 1 to 10 carbon atoms (C₁₋₁₀), in certain embodiments from 1 to 8 carbon atoms (C₁₋₈), in certain embodiments, from 1 to 6 carbon atoms (C₁₋₆), in certain embodiments from 1 to 4 carbon atoms (C₁₋₄), and in certain embodiments, from 1 to 3 carbon atoms (C₁₋₃).

“Acyl” by itself or as part of another substituent refers to a radical —C(O)R⁷⁰, where R⁷⁰ is hydrogen, alkyl, heteroalkyl, cycloalkyl, heterocycloalkyl, cycloalkylalkyl, heterocycloalkylalkyl, aryl, heteroaryl, arylalkyl, or heteroarylalkyl, which can be substituted, as defined herein. Examples of acyl groups include, but are not limited to, formyl, acetyl, cyclohexylcarbonyl, cyclohexylmethylcarbonyl, benzoyl, benzylcarbonyl, and the like.

“Alkoxy” by itself or as part of another substituent refers to a radical —OR⁷¹ where R⁷¹ is alkyl, cycloalkyl, cycloalkylalkyl, aryl, or arylalkyl, which can be substituted, as defined herein. Examples of alkoxy groups include, but are not limited to, methoxy, ethoxy, propoxy, butoxy, cyclohexyloxy, and the like. In certain embodiments, an alkoxy group is C₁₋₁₈ alkoxy, in certain embodiments, C₁₋₁₂ alkoxy, in certain embodiments, C₁₋₆ alkoxy, in certain embodiments, C₁₋₄ alkoxy, and in certain embodiments, C₁₋₃ alkoxy. “Alkoxycarbonyl” by itself or as part of another substituent refers to a radical —C(O)OR⁷² where R⁷² represents an alkyl, as defined herein. Examples of alkoxycarbonyl groups include, but are not limited to, methoxycarbonyl, ethoxycarbonyl, propoxycarbonyl, and butoxycarbonyl, and the like.

“Amino” refers to the radical —NH₂.

“Anesthesia” as used herein includes general anesthesia and deep sedation. General anesthesia is a drug-induced loss of consciousness during which patients are not arousable, even by painful stimulation. Deep sedation is a drug-induced depression of consciousness during which patients cannot be easily aroused but respond purposefully following repeated or painful stimulation. Reflex withdrawal from a painful stimulus is not a purposeful response. In deep sedation the ability of a patient to maintain ventilatory function may be impaired, while in general anesthesia, the ability to independently maintain ventilatory function is often impaired and often requires intervention in maintaining an open airway.

“Aryl” by itself or as part of another substituent refers to a monovalent aromatic hydrocarbon radical derived by the removal of one hydrogen atom from a single carbon atom of a parent aromatic ring system. Aryl encompasses benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can have from 6 to 20 carbon atoms (C₆₋₂₀), from 6 to 12 carbon atoms (C₆₋₁₂), and in certain embodiments, from 6 to 10 carbon atoms (C₆₋₁₀). Aryl, however, does not encompass or overlap in any way with heteroaryl, separately defined herein.

Aryl, however, does not encompass or overlap in any way with heteroaryl, separately defined herein. Hence, a multiple ring system in which one or more carbocyclic aromatic rings is fused to a heterocycloalkyl aromatic ring, is heteroaryl, not aryl, as defined herein.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl, or alkynyl moiety of the arylalkyl group is C₁₋₁₀ and the aryl moiety is C₆₋₂₀, and in certain embodiments, an arylalkyl group is C₇₋₂₀ arylalkyl, e.g., the alkanyl, alkenyl, or alkynyl moiety of the arylalkyl group is C₁₋₈ and the aryl moiety is C₆₋₁₂.

“AUC” is the area under a curve representing the concentration of a compound in a biological fluid in a patient as a function of time following administration of the compound to the patient. Examples of biological fluids include plasma and blood. The AUC can be determined by measuring the concentration of a compound in a biological fluid such as the plasma or blood using methods such as liquid chromatography-tandem mass spectrometry (LC/MS/MS), at various time intervals, and calculating the area under the plasma concentration-versus-time curve. Suitable methods for calculating the AUC from a drug concentration-versus-time curve are well known in the art. As relevant to the disclosure herein, an AUC for propofol can be determined by measuring the concentration of propofol in the plasma or blood of a patient following oral administration of a dosage form comprising a propofol prodrug.

“Bioavailability” refers to the rate and amount of a drug that reaches the systemic circulation of a patient following administration of the drug or prodrug thereof to the patient and can be determined by evaluating, for example, the plasma or blood concentration-versus-time profile for a drug. Parameters useful in characterizing a plasma or blood concentration-versus-time curve include the area under the curve (AUC), the time to peak concentration (Tₘₐₓ), and the maximum drug concentration (Cₘₐₓ), where Cₘₐₓ is the maximum concentration of a drug in the plasma or blood of a patient following administration of a dose of the drug or form of drug to the patient, and Tₘₐₓ is the time to the maximum concentration (Cₘₐₓ) of a drug in the plasma or blood of a patient following administration of a dose of the drug or form of drug to the patient.

“Cₘₐₓ” is the highest drug concentration observed in the plasma or blood following a dose of drug.

Compounds encompassed by structural Formulae (I)-(IV) disclosed herein include any specific compounds within these formulae. Compounds may be identified either by their chemical structure and/or chemical name. When the chemical structure and chemical name conflict, the chemical structure is determinative of the identity of the compound. The compounds described herein may contain one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan.

Compounds of Formulae (I)-(IV) include, but are not limited to, optical isomers of compounds of Formulae (I)-(IV), racemates thereof, and other mixtures thereof. In such embodiments, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds of Formulae (I)-(IV) include Z- and E-forms (e.g., cis- and trans-forms) of compounds with double bonds.

In embodiments in which compounds of Formulae (I)-(IV) exist in various tautomeric forms, compounds of the present disclosure include all tautomeric forms of the compound. The compounds of Formulae (I)-(IV) may also exist in several tautomeric forms including the enol form, the keto form, and mixtures thereof. Accordingly, the chemical structures depicted herein encompass all possible tautomeric forms of the illustrated compounds. The compounds of Formulae (I)-(IV) also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated, or N-oxides. Certain compounds may exist in single or multiple crystalline or amorphous forms. In general, all physical forms are equivalent for the uses contemplated herein and are intended to be within the scope of the present disclosure.

Further, when partial structures of the compounds are illustrated, an asterisk (*) indicates the point of attachment of the partial structure to the rest of the molecule.

“Cycloalkoxycarbonyl” by itself or as part of another substituent refers to a radical —C(O)OR⁷⁶ where R⁷⁶ represents an cycloalkyl group as defined herein. Examples of cycloalkoxycarbonyl groups include, but are not limited to, cyclobutyloxycarbonyl, cyclohexyloxycarbonyl, and the like.

“Cycloalkyl” by itself or as part of another substituent refers to a saturated or partially saturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, and in certain embodiments, C₃₋₁₂ cycloalkyl or C₅₋₁₂ cycloalkyl.

“Cycloalkylalkyl” by itself or as part of another substituent refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₇₋₃₀ cycloalkylalkyl, e.g., the alkanyl, alkenyl, or alkynyl moiety of the cycloalkylalkyl group is C₁₋₁₀ and the cycloalkyl moiety is C₆₋₂₀, and in certain embodiments, a cycloalkylalkyl group is C₇₋₂₀ cycloalkylalkyl, e.g., the alkanyl, alkenyl, or alkynyl moiety of the cycloalkylalkyl group is C₁₋₈ and the cycloalkyl moiety is C₄₋₂₀ or C₆₋₁₂.

“Disease” refers to a disease, disorder, condition, or symptom of any of the foregoing.

“Dosage form” means a pharmaceutical composition in a medium, carrier, vehicle, or device suitable for administration to a patient.

“Halogen” refers to a fluoro, chloro, bromo, or iodo group.

“Heteroalkyl” by itself or as part of another substituent refer to an alkyl group in which one or more of the carbon atoms (and any associated hydrogen atoms) are independently replaced with the same or different heteroatomic groups. In some embodiments, heteroalkyl groups have from 1 to 8 carbon atoms. Examples of heteroatomic groups include, but are not limited to, —O—, —S—, —O—O—, —S—S—, —O—S—, —NR⁷⁷R⁷⁸—, ═N—N═, —N═N—, —N═N—NR⁷⁹R⁸⁰, —PR⁸¹—, —P(O)₂—, —POR⁸²—, —O—P(O)₂—, —SO—, —SO₂—, —SnR⁸³R⁸⁴— and the like, where R⁷⁷, R⁷⁸, R⁷⁹, R⁸⁰, R⁸¹, R⁸², R⁸³, and R⁸⁴ are independently hydrogen, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, or substituted heteroarylalkyl. Where a specific level of saturation is intended, the nomenclature “heteroalkanyl,” “heteroalkenyl,” or “heteroalkynyl” is used. In certain embodiments, each of R⁷⁷, R⁷⁸, R⁷⁹, R⁸⁰, R⁸¹, R⁸², R⁸³, and R⁸⁴ is independently chosen from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, or substituted C₇₋₁₈ heteroarylalkyl. Reference to, for example, a C₁₋₆ heteroalkyl, means a C₁₋₆ alkyl group in which at least one of the carbon atoms (and certain associated hydrogen atoms) is replaced with a heteroatom. For example C₁₋₆ heteroalkyl includes groups having five carbon atoms and one heteroatom, groups having four carbon atoms and two heteroatoms, etc. In certain embodiments, each R¹⁰ is independently chosen from hydrogen and C₁₋₃ alkyl. In certain embodiments, a heteroatomic group is chosen from —O—, —S—, —NH—, —N(CH₃)—, and —SO₂—.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one aromatic ring fused to at least one other ring, which can be aromatic or non-aromatic in which at least one ring atom is a heteroatom. Heteroaryl encompasses 5- to 12-membered aromatic, such as 5- to 7-membered, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heterocycloalkyl, aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. In certain embodiments, the total number of heteroatoms in the heteroaryl group is not more than two. Heteroaryl does not encompass or overlap with aryl as defined herein.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, and in certain embodiments from 5- to 12-membered heteroaryl or from 5- to 10-membered heteroaryl. In certain embodiments heteroaryl groups are those derived from thiophene, pyrrole, benzothiophene, benzofuran, indole, pyridine, quinoline, imidazole, oxazole, and pyrazine. In certain embodiments, a heteroaryl group is from 4- to 20-membered heteroaryl (C₄₋₂₀), and in certain embodiments from 4- to 12-membered heteroaryl (C₄₋₁₀). In certain embodiments, heteroaryl groups are those derived from thiophene, pyrrole, benzothiophene, benzofuran, indole, pyridine, quinoline, imidazole, oxazole, or pyrazine. For example, in certain embodiments, C₅ heteroaryl can be furyl, thienyl, pyrrolyl, imidazolyl, pyrazolyl, isothiazolyl, isoxazolyl

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, or heteroarylalkynyl is used. In certain embodiments, a heteroarylalkyl group is a 6- to 30-membered heteroarylalkyl, e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl.

“Heterocycloalkyl” by itself or as part of another substituent refers to a partially saturated or unsaturated cyclic alkyl radical in which one or more carbon atoms (and any associated hydrogen atoms) are independently replaced with the same or different heteroatom. Examples of heteroatoms to replace the carbon atom(s) include, but are not limited to, N, P, O, S, Si, etc. Where a specific level of saturation is intended, the nomenclature “heterocycloalkanyl” or “heterocycloalkenyl” is used. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like.

“Heterocycloalkylalkyl” by itself or as part of another substituent refers to an acyclic alkyl radical in which one of the hydrogen atoms bonded to a carbon atom, typically a terminal or sp³ carbon atom, is replaced with a heterocycloalkyl group. Where specific alkyl moieties are intended, the nomenclature heterocycloalkylalkanyl, heterocycloalkylalkenyl, or heterocycloalkylalkynyl is used. In certain embodiments, a heterocycloalkylalkyl group is a 6- to 30-membered heterocycloalkylalkyl, e.g., the alkanyl, alkenyl, or alkynyl moiety of the heterocycloalkylalkyl is 1- to 10-membered and the heterocycloalkyl moiety is a 5- to 20-membered heterocycloalkyl, and in certain embodiments, 6- to 20-membered heterocycloalkylalkyl, e.g., the alkanyl, alkenyl, or alkynyl moiety of the heterocycloalkylalkyl is 1- to 8-membered and the heterocycloalkyl moiety is a 5- to 12-membered heterocycloalkyl.

“Hydroxyl” refers to the group —OH.

“Parent aromatic ring system” refers to an unsaturated cyclic or polycyclic ring system having a conjugated π (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to a parent aromatic ring system in which one or more carbon atoms (and any associated hydrogen atoms) are independently replaced with the same or different heteroatom. Examples of heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Patient” refers to a mammal, for example, a human.

“Pharmaceutical composition” refers to at least one compound such as a compound of Formulae (I)-(IV), compound (1), or compound (2) and a pharmaceutically acceptable vehicle with which the compound is administered to a patient.

“Pharmaceutically acceptable” refers to approved or approvable by a regulatory agency of the Federal or a state government or listed in the U.S. Pharmacopeia or other generally recognized pharmacopeia for use in animals, and more particularly in humans.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like. In certain embodiments, a pharmaceutically acceptable salt is the hydrochloride salt.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound of the present disclosure can be administered to a patient and which does not destroy the pharmacological activity thereof and which is nontoxic when administered in doses sufficient to provide a therapeutically effective amount of the compound.

“Prodrug” refers to a derivative of a drug molecule that requires a transformation within the body to release the active drug. Prodrugs are frequently, although not necessarily, pharmacologically inactive until converted to the parent drug. Prodrugs can be obtained by bonding a promoiety (defined herein) typically via a functional group, to a drug. For example, referring to compounds of Formulae (I)-(IV), the promoiety is bonded to propofol via the hydroxyl group of the drug.

“Prodrug of propofol” refers to a compound in which a promoiety that is cleavable in vivo, and is covalently bound to the propofol molecule. In certain embodiments, a prodrug may be actively transported by transporters expressed in the enterocytes lining the gastrointestinal tract such as, for example, the PEPT1 transporter. Propofol prodrugs can be stable in the gastrointestinal tract and following absorption are cleaved in the systemic circulation to release propofol. In certain embodiments, a prodrug of propofol provides a greater oral bioavailability of propofol compared to the oral bioavailability of propofol when administered as a uniform liquid immediate release formulation. In certain embodiments, a prodrug of propofol provides a high oral bioavailability of propofol, for example, exhibiting a propofol oral bioavailability that is at least 10 times greater than the oral bioavailability of propofol when orally administered in an equivalent dosage form. In certain embodiments, a prodrug of propofol is a compound having a structure encompassed by any one of Formulae (I)-(IV), compound (1), and/or compound (2), pharmaceutically acceptable salts thereof, or pharmaceutically acceptable solvates of any of the foregoing, infra. In certain embodiments, a propofol prodrug is compound (2), a pharmaceutically acceptable salt thereof, or a pharmaceutically acceptable solvate of any of the foregoing.

“Promoiety” refers to a chemical group, i.e. moiety, bonded to a drug, typically to a functional group of the drug, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. Cleavage of the promoiety may proceed spontaneously, such as via a hydrolysis reaction, or may be catalyzed or induced by another agent, such as by an enzyme, by light, by acid, or by a change of or exposure to a physical or environmental parameter such as a change of temperature, pH, etc. The agent may be endogenous to the conditions of use, such as an enzyme present in the systemic circulation of a patient to which the prodrug is administered or the acidic conditions of the stomach, or the agent may be supplied exogenously. As an example, for a prodrug of Formula (IV), the promoiety is:

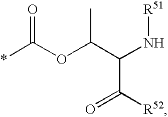

where R⁵¹ and R⁵² are as defined herein, and the drug is propofol.

“Sedation” as used herein refers to minimal sedation and/or moderate sedation (see e.g., American Society of Anesthesiologists, Anesthesiology 2002, 96, 1004-17). Minimal sedation, also referred to as anxiolysis, is a minimally depressed level of consciousness that retains the patient's ability to independently and continuously maintain an airway and respond appropriately to physical stimulation or verbal command that is produced by a pharmacological or non-pharmacological method or combination thereof. Although cognitive function and coordination may be modestly impaired, ventilatory and cardiovascular functions are unaffected. When the intent is minimal sedation in adults, the appropriate dosing is no more than the maximum recommended dose that can be prescribed for unmonitored home use, e.g., a maximum recommended therapeutic dose. Moderate sedation is a drug-induced depression of consciousness during which patients respond purposefully to verbal commands, either alone or accompanied by light tactile stimulation. No intervention is required to maintain a patient's airway. Sedation is a continuum and it is not always possible to predict how an individual patient will respond. A sedative dose can be determined by incremental dosing, administering multiple doses of a drug, such as a propofol prodrug provided by the present disclosure, until a desired effect is reached. A variety of scales can be used to assess sedation including, for example, the Ramsay scale (Ramsay et al., Br Med J 1974, 2, 656-659), and the Observer's Assessment of Alertness/Sedation scale (Chernik et al., J Clin Psychopharmacol 1990, 10, 244-251), and others (see e.g., Sessler, Chest 2004, 126, 1727-1730). Objective measures of sedation include measurement of electroencephalogram parameters such as the Bispectral Index version XP and the Patient State Analyzer (see e.g., Chisholm et al., Mayo Clin Proc 2006, 81(1), 46-52; and Tonneri et al., Best Pract Res Clin Anaesthesiol 2006, 20(1), 191-2000). In certain embodiments, sedation refers to minimal sedation, and in certain embodiments, moderate sedation.

“Solvate” refers to a molecular complex of a compound with one or more solvent molecules in a stoichiometric or non-stoichiometric amount. Such solvent molecules are those commonly used in the pharmaceutical art, which are known to be innocuous to recipient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a complex where the one or more solvent molecules are water including monohydrates and hemi-hydrates.

“Substantially one diastereomer” refers to a compound containing two or more stereogenic centers such that the diastereomeric excess (d.e.) of the compound is greater than or about at least 90%. In certain embodiments, the d.e. is, for example, greater than or at least about 91%, about 92%, about 93%, about 94%, about 95%, about 96%, about 97%, about 98%, or about 99%.

“Substituted” refers to a group in which one or more hydrogen atoms are independently replaced with the same or different substituent(s). Examples of substituents include, but are not limited to, -Q, —R⁶⁰, —O⁻, (—OH), ═O, —OR⁶⁰, —SR⁶⁰, —S⁻, ═S, —NR⁶⁰R⁶¹, ═NR⁶⁰, —CX₃, —CN, —CF₃, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹, —C(NR⁶²)NR⁶⁰R⁶¹, —S(O)₂, NR⁶⁰R⁶¹, —NR⁶³S(O)₂R⁶⁰, —NR⁶³C(O)R⁶⁰, and —S(O)R⁶⁰ where each Q is independently a halogen; each R⁶⁰ and R⁶¹ are independently chosen from hydrogen, alkyl, substituted alkyl, alkoxy, substituted alkoxy, cycloalkyl, substituted cycloalkyl, heterocycloalkyl, substituted heterocycloalkyl, aryl, substituted aryl, heteroaryl, substituted heteroaryl, arylalkyl, substituted arylalkyl, heteroarylalkyl, and substituted heteroarylalkyl, or R⁶⁰ and R⁶¹ together with the nitrogen atom to which they are bonded form a ring chosen from a heterocycloalkyl, substituted heterocycloalkyl, heteroaryl, and substituted heteroaryl ring, and R⁶² and R⁶³ are independently chosen from hydrogen, alkyl, substituted alkyl, aryl, substituted aryl, arylalkyl, substituted arylalkyl, cycloalkyl, substituted cycloalkyl, heterocycloalkyl, substituted heterocycloalkyl, heteroaryl, substituted heteroaryl, heteroarylalkyl, and substituted heteroarylalkyl, or R⁶² and R⁶³ together with the atom to which they are bonded form a ring chosen from a heterocycloalkyl, substituted heterocycloalkyl, heteroaryl, and substituted heteroaryl ring. In certain embodiments, a tertiary amine or aromatic nitrogen may be substituted with one or more oxygen atoms to form the corresponding nitrogen oxide.

In certain embodiments, substituted aryl and substituted heteroaryl include one or more of the following substitute groups: F, Cl, Br, C₁₋₃ alkyl, substituted alkyl, C₁₋₃ alkoxy, —S(O)₂NR⁶⁰R⁶¹, —NR⁶⁰R⁶¹, —CF₃, —OCF₃, —CN, —NR⁶⁰S(O)₂R⁶¹, —NR⁶⁰C(O)R⁶¹, C₅₋₁₀ aryl, substituted C₅₋₁₀ aryl, C₅₋₁₀ heteroaryl, substituted C₅₋₁₀ heteroaryl, —C(O)OR⁶⁰, —NO₂, —C(O)R⁶⁰, —C(O)NR⁶⁰R⁶¹, —OCHF₂, C₁₋₃ acyl, —SR⁶⁰, —S(O)₂OH, —S(O)₂R⁶⁰, —S(O)R⁶⁰, —C(S)R⁶⁰, —C(O)O⁻, —C(S)OR⁶⁰, —NR⁶⁰C(O)NR⁶¹R⁶², —NR⁶⁰C(S)NR⁶¹R⁶², and —C(NR⁶⁰)NR⁶¹R⁶², C₃₋₈ cycloalkyl, and substituted C₃₋₈ cycloalkyl, wherein R⁶⁰R⁶¹, and R⁶² are independently chosen from hydrogen and C₁₋₄ alkyl.

In certain embodiments, each substituent group can independently be chosen from halogen, —NO₂, —OH, —COOH, —NH₂, —CN, —CF₃, —OCF₃, C₁₋₈ alkyl, substituted C₁₋₈ alkyl, C₁₋₈ alkoxy, and substituted C₁₋₈ alkoxy.

“Controlled delivery” means continuous or discontinuous release of a compound over a prolonged period of time, wherein the compound is released at a controlled rate over a controlled period of time in a manner that provides for upper gastrointestinal and lower gastrointestinal tract delivery, coupled with improved compound absorption as compared to the absorption of the compound in an immediate release oral dosage form.

“Sustained release” refers to release of a therapeutic amount of a drug, a prodrug, or an active metabolite of a prodrug over a period of time that is longer than that of a conventional formulation of the drug, e.g. an immediate release formulation of the compound. For oral formulations, the term “sustained release” typically means release of the compound within the gastrointestinal tract lumen over a time period from about 2 to about 30 hours, and in certain embodiments, over a time period from about 4 to about 24 hours. Sustained release formulations achieve therapeutically effective concentrations of the drug in the systemic circulation over a prolonged period of time relative to that achieved by oral administration of an immediate release formulation of the drug. “Delayed release” refers to release of a drug, a prodrug, or an active metabolite of a prodrug into the gastrointestinal lumen after a delayed time period, for example a delay of about 1 to about 12 hours, relative to that achieved by oral administration of an immediate release formulation of the drug.

“Treating” or “treatment” of any disease or disorder refers to reversing, alleviating, arresting or ameliorating a disease, disorder, or at least one of the clinical symptoms of a disease or disorder, reducing the risk of acquiring a disease, disorder, or at least one of the clinical symptoms of a disease or disorder, inhibiting or delaying the progress of a disease, disorder or at least one of the clinical symptoms of the disease or disorder, or reducing the risk of developing a disease, disorder, or at least one of the clinical symptoms of a disease or disorder. “Treating” or “treatment” also refers to inhibiting the disease, disorder, or at least one of the clinical symptoms of a disease or disorder, either physically, (e.g., stabilization of a discernible symptom), physiologically, (e.g., stabilization of a physical parameter), or both, and to inhibiting at least one physical parameter which may or may not be discernible to the patient. In certain embodiments, “treating” or “treatment” refers to delaying the onset of the disease or disorder or at least one or more symptoms thereof in a patient which may be exposed to or predisposed to a disease or disorder even though that patient does not yet experience or display symptoms of the disease or disorder.

“Therapeutically effective amount” refers to the amount of a compound that, when administered to a patient for treating a disease or disorder, or at least one of the clinical symptoms of a disease or disorder, is sufficient to affect such treatment of the disease, disorder, or symptom. The “therapeutically effective amount” can vary depending, for example, on the compound, the disease, disorder, and/or symptoms of the disease or disorder, severity of the disease, disorder, and/or symptoms of the disease or disorder, the age, weight, and/or health of the patient to be treated, and the judgment of the prescribing physician. An appropriate therapeutically effective amount in any given instance may be ascertained by those skilled in the art or capable of determination by routine experimentation.

“Therapeutically effective dose” refers to a dose of a drug, prodrug or active metabolite of a prodrug that provides effective treatment of a disease or disorder in a patient. A therapeutically effective dose may vary from compound to compound and from patient to patient, and may depend upon factors such as the condition of the patient and the route of delivery. A therapeutically effective dose may be determined in accordance with routine pharmacological procedures known to those skilled in the art.

Reference is now made in detail to embodiments of the present disclosure. The disclosed embodiments are not intended to be limiting of the claims. To the contrary, the claims are intended to cover alternatives, modifications, and equivalents.

### Propofol Prodrugs

In certain embodiments, propofol prodrugs provide an oral bioavailability of propofol that is at least 10 times greater than the oral bioavailability of propofol when orally administered in an equivalent dosage form. In certain embodiments, propofol prodrugs provide an oral bioavailability of propofol that is at least 10 times greater than the oral bioavailability of propofol provided by propofol when orally administered to a patient as a uniform liquid immediate release formulation.

Propofol prodrugs include prodrugs, conjugates, and complexes in which propofol is attached to at least one moiety. The moiety covalently or non-covalently attached to propofol may enhance permeability through gastrointestinal epithelia via passive and/or active transport mechanisms, may control the release of propofol in the gastrointestinal tract, and/or may inhibit enzymatic and chemical degradation of propofol in the gastrointestinal tract. For propofol prodrugs in which the moiety remains attached to the propofol molecule after absorption, the moiety may enhance permeability through other biological membranes, and/or can inhibit enzymatic and chemical degradation of propofol in the systemic circulation.

Reducing the rate of metabolism of a drug in the gastrointestinal tract and/or enhancing the rate by which a drug is absorbed from the gastrointestinal tract may enhance the oral bioavailability of a drug. An orally administered drug will pass through the gastrointestinal system in about 11 to 31 hours. In general, an orally ingested drug resides about 1 to 6 hours in the stomach, about 2 to 7 hours in the small intestine, and about 8 to 18 hours in the colon. The oral bioavailability of a particular drug will depend on a number of factors including the residence time in a particular region of the gastrointestinal tract, the rate the drug is metabolized within the gastrointestinal tract, the rate at which a drug is metabolized in the systemic circulation, and the rate by which the compound is absorbed from a particular region or regions of the gastrointestinal tract, which include passive and active transport mechanisms. Several methods have been developed to achieve these objectives, including drug modification, incorporating the drug or modified drug in a controlled release or sustained release dosage form, and/or by co-administering adjuvants, which can be incorporated in the dosage form containing the active compound.

Examples of propofol prodrugs that provide a high oral bioavailability of propofol include bile acid prodrugs, peptide conjugates, and prodrugs in which propofol is bonded to an amino acid or small peptide via a linkage. Prodrugs are compounds in which a promoiety is typically covalently bonded to a drug. Following absorption from the gastrointestinal tract, the promoiety is cleaved to release the drug into the systemic circulation. While in the gastrointestinal tract, the promoiety can protect the drug from the harsh chemical environment, and can also facilitate absorption. Promoieties can be designed, for example, to enhance passive absorption, e.g., lipophilic promoieties, and/or to enhance absorption via active transport mechanisms, e.g., substrate promoieties. In particular, active transporters differentially expressed in regions of the gastrointestinal tract may be preferentially targeted to enhance absorption. For example, a propofol prodrug may incorporate a promoiety that is a substrate of the PEPT1 transporter expressed in the small intestine. Zerangue et al., U.S. Pat. No. 6,955,888 and U.S. Application Publication No. 2005/0214853, each of which is incorporated by reference herein in its entirety, disclose methodologies for screening drugs, conjugates or conjugate moieties, linked or linkable to drugs, for their capacity to be transported as substrates via the PEPT1 and PEPT2 transporters, which are known to be expressed in the human small intestine (see, e.g., Fei et al., Nature 1964, 386, 563-566; and Miyamoto et al., Biochimica et Biophysica Acta 1996, 1305, 34-38). Zerangue et al., U.S. Application Publication No. 2003/0158254 also disclose several transporters expressed in the human colon including the sodium dependent multi-vitamin transporter (SMVT) and monocarboxylate transporters MCT1 and MCT4, and methods of identifying agents, or conjugate moieties that are transporter substrates, and agents, conjugates, and conjugate moieties that may be screened for substrate activity. Zerangue et al. further disclose compounds that may be screened and are variants of known transporter substrates such as bile salts or acids, steroids, ecosanoids, or natural toxins or analogs thereof, as described by Smith, Am. J Physiol 1987, 223, 974-978; Smith, Am J Physiol 1993, 252, G479-G484; Boyer, Proc Natl Acad Sci USA 1993, 90, 435-438; Fricker, Biochem J 1994, 299, 665-670; Ficker, Biochem J 1994, 299, 665-670; and Ballatori et al., Am J Physiol 2000, 278, G57-G63, as well as the linkage of drugs to conjugate moieties.

These prodrugs, which can provide enhanced oral bioavailability of propofol, are distinguishable from propofol prodrugs having promoieties that provide enhanced aqueous solubility of propofol for intravenous administration. Propofol exhibits poor aqueous solubility and it is desirable that intravenously administered drugs be water-soluble. Propofol is widely used as a hypnotic sedative for intravenous administration in the induction and maintenance of anesthesia or sedation in humans and animals. Propofol prodrugs with enhanced aqueous solubility for intravenous administration are disclosed, for example, by Stella et al., U.S. Pat. Nos. 6,204,257 and 6,872,838, and U.S. Pat. No. 7,244,718; Marappan et al., U.S. Pat. No. 7,250,412; and Wingard et al., U.S. Application Publication No. 2005/0203068.

Examples of propofol prodrugs capable of providing an increased oral bioavailability of propofol in which propofol is bonded to an amino acid or small peptide via a linkage are disclosed in Gallop et al., U.S. Pat. Nos. 7,220,875 and 7,230,003 and U.S. Application Publication No. 2006/0287525; Xu et al., U.S. Pat. No. 7,241,807; Xu et al., U.S. Application Publication Nos. 2006/0100160, and 2006/0205969, and U.S. application Ser. No. 11,923,444 filed Oct. 24, 2007, each of which is incorporated by reference herein in its entirety.

In certain embodiments, prodrugs of propofol may be chosen from any of the genuses or species of compounds of Formula (I) as disclosed in Gallop et al., U.S. Pat. No. 7,220,875:

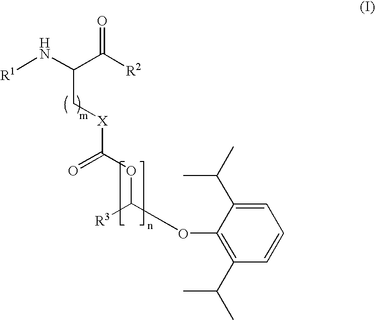

a pharmaceutically acceptable salt thereof, and a pharmaceutically acceptable solvate of any of the foregoing, wherein:

X is chosen from a bond, —CH₂—, —NR¹¹—, —O—, and —S—;

m is chosen from 1 and 2;

n is chosen from 0 and 1;

R¹ is chosen from hydrogen, [R⁵NH(CHR⁴)ₚC(O)]—, R⁶—, R⁶C(O)—, and R⁶OC(O)—;

R² is chosen from —OR⁷ and —[NR⁸(CHR⁹)_{q}C(O)OR⁷];

p and q are independently chosen from 1 and 2;

each R³ is independently chosen from hydrogen, alkyl, substituted alkyl, alkoxycarbonyl, aryl, substituted aryl, arylalkyl, carbamoyl, substituted carbamoyl, cycloalkyl, substituted cycloalkyl, heterocycloalkyl, heteroaryl, substituted heteroaryl, and heteroarylalkyl;

each R⁴ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, or when R⁴ and R⁵ are attached to adjacent atoms then R⁴ and R⁵ together with the atoms to which they are bonded form a ring chosen from a heterocycloalkyl and substituted heterocycloalkyl ring;

R⁵ is chosen from hydrogen, R⁶—, R⁶C(O)—, and R⁶OC(O)—;

R⁶ is chosen from alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;

R⁷ is chosen from hydrogen, alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;

R⁸ is chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl;

each R⁹ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, or when R⁸ and R⁹ are attached to adjacent atoms then R⁸ and R⁹ together with the atoms to which they are bonded form a ring chosen from a heterocycloalkyl and substituted heterocycloalkyl ring; and

R¹¹ is chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl;

with the provisos that:

when R¹ is [R⁵NH(CHR⁴)ₚC(O)]— then R² is —OR⁷; and

when R² is —[NR⁸(CHR⁹)_{q}C(O)OR⁷] then R¹ is not [R⁵NH(CHR⁴)ₚC(O)]—.

In certain embodiments, prodrugs of propofol may be chosen from any of the genuses or species of compounds of Formula (II) as disclosed in Gallop et al., U.S. Pat. No. 7,230,003:

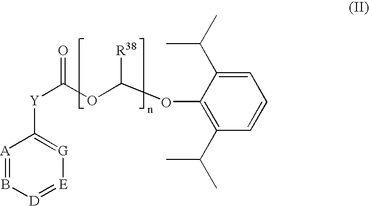

a pharmaceutically acceptable salt thereof, and a pharmaceutically acceptable solvate of any of the foregoing, wherein:

n is chosen from 0 and 1;

Y is chosen from a bond, CR²¹R²², NR²³, O, and S;

A is chosen from CR²⁴ and N;

B is chosen from CR²⁵ and N;

D is chosen from CR²⁶ and N;

E is chosen from CR²⁷ and N;

G is chosen from CR²⁸ and N;

R³⁸ is chosen from hydrogen, alkyl, substituted alkyl, alkoxycarbonyl, aryl, substituted aryl, arylalkyl, carbamoyl, substituted carbamoyl, cycloalkyl, substituted cycloalkyl, heterocycloalkyl, heteroaryl, substituted heteroaryl, and heteroarylalkyl;

R²¹ and R²² are independently chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl;

R²³ is chosen from hydrogen, alkyl, substituted alkyl, aryl, arylalkyl, cycloalkyl, and heteroaryl;

R²⁴ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;

R²⁵ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;

R²⁶ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;

R²⁷ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;

R²⁸ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;

W is chosen from a bond, —CR³²R³³—NR³⁴, O, and S;

Z is chosen from —CR³⁵R³⁶, —NR³⁷, O, and S;

k is chosen from 0 and 1;

r is chosen from 1, 2, and 3;

each of R²⁹, R³⁰, R³¹, R³², R³³, R³⁵, and R³⁶ is independently chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl; and

R³⁴ and R³⁷ are independently chosen from hydrogen, alkyl, substituted alkyl, aryl, arylalkyl, cycloalkyl, and heteroaryl;

with the provisos that:

at least one of A, B, D, E, and G is not N;

one and only one of R²⁴, R²⁵, R²⁶, R²⁷, or R²⁸ is —W[C(O)]ₖZ(CR²⁹R³⁰)ᵣCO₂R³¹; and

if k is 0 then W is a bond.

In certain embodiments, prodrugs of propofol may be chosen from any of the genuses or species of compounds of Formula (III) as disclosed in Xu et al., U.S. Pat. No. 7,241,807:

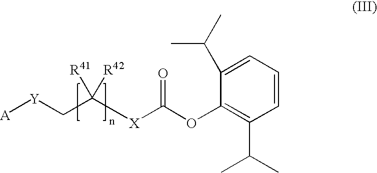

a pharmaceutically acceptable salt thereof, and a pharmaceutically acceptable solvate of any of the foregoing, wherein:

each R⁴¹ and R⁴² is independently chosen from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or R⁴¹ and R⁴² together with the carbon atom to which they are bonded form a ring chosen from a cycloalkyl, substituted cycloalkyl, heterocycloalkyl, and substituted heterocycloalkyl ring;

A is chosen from hydrogen, acyl, substituted acyl, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl; or A, Y, and one of R⁴¹ and R⁴² together with the atoms to which they are bonded form a ring chosen from a heterocycloalkyl and substituted heterocycloalkyl ring;

Y is chosen from —O— and —NR⁴³—;

R⁴³ is chosen from hydrogen, alkyl, substituted alkyl, arylalkyl, and substituted arylalkyl;

n is an integer from 1 to 5;

X is chosen from —NR⁴⁴—, —O—, —CH₂, and —S—; and

R⁴⁴ is chosen from hydrogen, alkyl, substituted alkyl, arylalkyl, and substituted arylalkyl.

In certain embodiments, prodrugs of propofol may be chosen from any of the genuses or species of compounds of Formula (IV) as disclosed in Xu et al., U.S. Application Publication No. 2006/0100160:

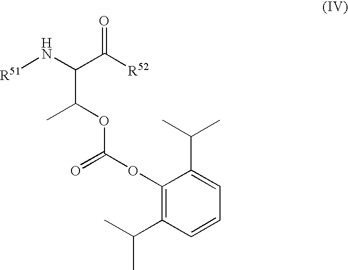

a pharmaceutically acceptable salt thereof, and a pharmaceutically acceptable solvate of any of the foregoing, wherein:

R⁵¹ is chosen from hydrogen, [R⁵⁵NH(CHR⁵⁴)ₚC(O)]—, R⁵⁶—, R⁵⁶C(O)—, and R⁵⁶OC(O)—;

R⁵² is chosen from —OR⁵⁷ and —[NR⁵⁸(CHR⁵⁹)_{q}C(O)OR⁵⁷];

p and q are independently chosen from 1 and 2;

each R⁵⁴ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, or when R⁵⁴ and R⁵⁵ are bonded to adjacent atoms then R⁵⁴ and R⁵⁵ together with the atoms to which they are bonded form a ring chosen from a heterocycloalkyl and substituted heterocycloalkyl ring;

R⁵⁵ is chosen from hydrogen, R⁵⁶—, R⁵⁶C(O)—, and R⁵⁶OC(O)—;

R⁵⁶ is chosen from alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;

R⁵⁷ is chosen from hydrogen, alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;

R⁵⁸ is chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl; and

each R⁵⁹ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, or when R⁵⁸ and R⁵⁹ are bonded to adjacent atoms then R⁵⁸ and R⁵⁹ together with the atoms to which they are bonded form a ring chosen from a heterocycloalkyl and substituted heterocycloalkyl ring;

with the proviso that when R⁵² is —[NR⁵⁸(CHR⁵⁹)_{q}C(O)OR⁵⁷] then R⁵¹ is not [R⁵⁵NH(CHR⁵⁴)ₚC(O)]—.

In certain embodiments, a prodrug of propofol is 2-amino-3-methyl-3-(2,6-diisopropyl-phenoxycarbonyloxy)-propanoic acid (1):

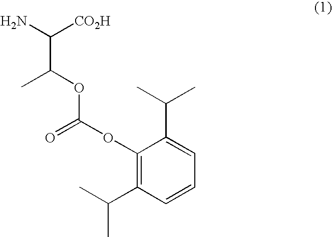

a pharmaceutically acceptable salt thereof, or a pharmaceutically acceptable solvate of any of the foregoing.

In certain embodiments of compound (1), the α-carbon of the amino acid residue is of the L-configuration. In certain embodiments of compound (1), the α-carbon of the amino acid residue is of the D-configuration.

In certain embodiments, a prodrug of propofol is (S)-2-amino-3-(2,6-diisopropyl-phenoxycarbonyloxy)-propanoic acid (2) as disclosed in Xu et al., U.S. Application Publication No. 2006/0205969:

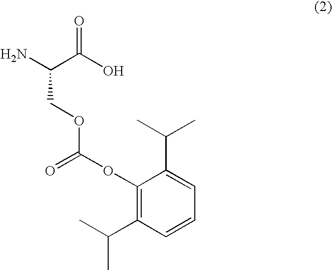

a pharmaceutically acceptable salt thereof, or a pharmaceutically acceptable solvate of any of the foregoing.

In certain embodiments, compound (2) is a crystalline form of 2-amino-3-(2,6-diisopropyl-phenoxycarbonyloxy)-propanoic acid, a pharmaceutically acceptable salt thereof, or a pharmaceutically acceptable solvate of any of the foregoing. In certain embodiments, a prodrug of propofol of Formula (2) is a crystalline form of (S)-2-amino-3-(2,6-diisopropylphenoxycarbonyloxy)-propanoic acid, a pharmaceutically acceptable salt thereof, or a pharmaceutically acceptable solvate of any of the foregoing. In certain embodiments, a prodrug of propofol is crystalline 2-amino-3-(2,6-diisopropylphenoxycarbonyloxy)-propanoic acid hydrochloride. In certain embodiments, a prodrug of propofol is crystalline (S)-2-amino-3-(2,6-diisopropylphenoxycarbonyloxy)-propanoic acid hydrochloride. In certain embodiments, a prodrug of propofol is crystalline (S)-2-amino-3-(2,6-diisopropylphenoxy-carbonyloxy)-propanoic acid hydrochloride having characteristic peaks (2θ) at 5.1°±0.2°, 9.7°±0.2°, 11.0°±0.2°, 14.1°±0.2°, 15.1°±0.2°, 15.8°±0.2°, 17.9°±0.2°, 18.5°±0.2°, 19.4°±0.2°, 20.1°±0.2°, 21.3°±0.2°, 21.7°±0.2°, 22.5°±0.2°, 23.5°±0.2°, 24.4°±0.2°, 25.1°±0.2°, 26.8°±0.2°, 27.3°±0.2°, 27.8°±0.2°, 29.2°±0.2°, 29.6°±0.2°, 30.4°±0.2°, and 33.4°±0.2° in an X-ray powder diffraction pattern measured using CuKα radiation. In certain embodiments, a prodrug of propofol is crystalline (S)-2-amino-3-(2,6-diisopropylphenoxycarbonyloxy)-propanoic acid hydrochloride having characteristic peaks (2θ) at 5.1°±0.2°, 9.7°±0.2°, 11.0°±0.2°, 14.1°±0.2°, 15.1°±0.2°, 15.8°±0.2°, 17.9°±0.2°, 18.5°±0.2°, 20.1°±0.2°, 22.5°±0.2°, 23.5°±0.2°, 25.1°±0.2°, 29.2°±0.2°, 29.6°±0.2°, and 33.4°±0.2° in an X-ray powder diffraction pattern measured using CuKα radiation.

In certain embodiments, a prodrug of propofol is crystalline (S)-2-amino-3-(2,6-diisopropylphenoxycarbonyloxy)-propanoic acid hydrochloride having a melting point from about 180° C. to about 200° C. In certain embodiments, a prodrug of propofol is crystalline (S)-2-amino-3-(2,6-diisopropylphenoxycarbonyloxy)-propanoic acid hydrochloride having a melting point from about 185° C. to about 195° C. In certain embodiments, a prodrug of propofol is crystalline (S)-2-amino-3-(2,6-diisopropylphenoxycarbonyloxy)-propanoic acid hydrochloride having a melting point from about 188° C. to about 189° C.

In certain embodiments, a prodrug of propofol is crystalline 2-amino-3-(2,6-diisopropylphenoxycarbonyloxy)-propanoic acid mesylate. In certain embodiments, a prodrug of propofol is crystalline (S)-2-amino-3-(2,6-diisopropylphenoxycarbonyloxy)-propanoic acid mesylate. In certain embodiments, a prodrug of propofol is crystalline (S)-2-amino-3-(2,6-diisopropylphenoxycarbonyloxy)-propanoic acid mesylate having characteristic peaks (2θ) at 4.2°±0.1°, 11.7°±0.1°, 12.1°±0.1°, 12.6°±0.1°, 16.8°±0.1°, 18.4°±0.2°, 21.0°±0.1°, 22.3°±0.1°, 22.8°±0.2°, 24.9°±0.2°, 25.3°±0.1°, 26.7°±0.2°, and 29.6°±0.1° in an X-ray powder diffraction pattern measured using CuKα radiation. In certain embodiments, a prodrug of propofol is crystalline (S)-2-amino-3-(2,6-diisopropylphenoxycarbonyloxy)-propanoic acid mesylate having characteristic peaks (2θ) at 4.2°±0.1°, 12.6°±0.1°, 16.8°±0.1°, 21.0°±0.1°, 25.3°±0.1°, 2 and 29.6°±0.1° in an X-ray powder diffraction pattern measured using CuKα radiation.

In certain embodiments, a prodrug of propofol is crystalline (S)-2-amino-3-(2,6-diisopropylphenoxycarbonyloxy)-propanoic acid mesylate having a melting point from about 156° C. to about 176° C. In certain embodiments, a prodrug of propofol is crystalline (S)-2-amino-3-(2,6-diisopropylphenoxycarbonyloxy)-propanoic acid mesylate having a melting point from about 161° C. to about 172° C. In certain embodiments, a prodrug of propofol is crystalline (S)-2-amino-3-(2,6-diisopropylphenoxycarbonyloxy)-propanoic acid mesylate having a melting point from about 166° C. to about 167° C.

Propofol prodrugs of Formulae (I)-(IV) may be administered orally and transported across cells (i.e., enterocytes) lining the lumen of the gastrointestinal tract. Certain of the compounds of structural Formulae (I)-(IV) may be substrates for the proton-coupled intestinal peptide transport system (PEPT1) (Leibach et al., Annu. Rev Nut. 1996, 16, 99-119), which mediates the cellular uptake of small intact peptides consisting of two or three amino acids that are derived from the digestion of dietary proteins. In the intestine, where small peptides are not effectively absorbed by passive diffusion, PEPT1 may act as a vehicle for the effective uptake of small peptides across the apical membrane of the gastric mucosa including propofol prodrugs of Formulae (I)-(IV).

Methods for determining whether propofol prodrugs of Formulae (I)-(IV) serve as substrates for the PEPT1 transporter are disclosed, for example, in Xu et al., U.S. Application Publication No. 2006/0100160. In vitro systems using cells engineered to heterologously express the PEPT1 transport system or cell-lines that endogenously express the transporter (e.g. Caco-2 cells) may be used to assay transport of compounds of Formulae (I)-(IV) by the PEPT1 transporter. Standard methods for evaluating the enzymatic conversion of a propofol prodrug to propofol in vitro are disclosed, for example, in Xu et al., U.S. Application Publication No. 2006/0100160.

Oral administration of propofol prodrugs to animals is described in Xu et al., U.S. Pat. No. 7,241,807 and U.S. Application Publication Nos. 2006/0100160, and 2006/0205969, which illustrate that propofol prodrugs can afford significant enhancement in the oral bioavailability of propofol relative to the oral bioavailability of propofol when administered as propofol in an equivalent dosage form. In certain embodiments, a prodrug of propofol provides greater than 10% absolute oral bioavailability of propofol, i.e., relative to the bioavailability of propofol following intravenous administration of an equimolar dose of propofol itself. A prodrug of propofol that provides at least about 10 times higher oral bioavailability of propofol compared to the oral bioavailability of propofol itself, and in certain embodiments, at least about 40 times higher oral bioavailability of propofol compared to the oral bioavailability of propofol itself when orally administered in an equivalent dosage form (see, e.g., Xu et al., U.S. Pat. No. 7,241,807 and U.S. Application Publication Nos. 2006/0041011, 2006/0100160, and 2006/0205969).

Methods of synthesizing prodrugs of propofol of Formula (I) are disclosed in Gallop et al., U.S. Pat. No. 7,220,875. Methods of synthesizing prodrugs of propofol of Formula (II) are disclosed in Gallop et al., U.S. Pat. No. 7,230,003. Methods of synthesizing prodrugs of propofol of Formulae (III) are disclosed in Xu et al., U.S. Pat. No. 7,241,807. Methods of synthesizing prodrugs of propofol of Formulae (IV) are disclosed in Xu et al., U.S. Application Publication No. 2006/0100160. Methods of synthesizing and crystallizing prodrugs of propofol of Formula (2) are disclosed in Xu et al., U.S. Application Publication No. 2006/0205969.

Propofol prodrugs of Formulae (I)-(IV) are distinguished from other propofol prodrugs by their ability to provide high oral bioavailability of propofol. Various prodrugs of propofol have been developed that enhance the aqueous solubility of propofol for intravenous administration (Stella et al., U.S. Pat. Nos. 6,204,257 and 6,872,838; Hendler et al., U.S. Pat. Nos. 6,254,853 and 6,362,234; Jenkins et al., U.S. Pat. No. 6,815,555; Wingard et al., U.S. Application Publication No. 2005/0203068; Marappan et al., U.S. Pat. No. 7,250,412; Orlando et al., U.S. Application Publication No. 2005/0267169; Fechner et al., Anesthesiology 2003, 99, 303-313; Tao et al., U.S. Application Publication No. 2007/0185217; Fechner et al., Anesthesiology 2004, 101, 626-639; Struys et al., Anesthesiology 2005, 103, 730-43; and Gibiansky et al., Anesthesiology 2005, 103, 718-729). While the use of such prodrugs for oral administration is disclosed, there is no evidence to suggest that any of the propofol prodrugs intended for use in aqueous intravenous formulations provides clinically relevant systemic propofol concentrations when orally administered. Recently, Slusher and Wozniak, U.S. Application Publication No. 2007/0202158 disclosed the enhanced oral bioavailability of the O-phosphonooxymethyl propofol disodium salt.

Any of the propofol prodrugs disclosed herein may exhibit sufficient stability to enzymatic and/or chemical degradation in the gastrointestinal tract resulting in enhanced oral bioavailability of the propofol prodrug and/or propofol metabolite. The propofol prodrugs may also exhibit enhanced passive and/or active gastrointestinal absorption compared to propofol.

### Pharmaceutical Compositions

Propofol prodrugs provided by the present disclosure may be formulated into pharmaceutical compositions for use in oral dosage forms to be administered to patients.

Pharmaceutical compositions comprise at least one propofol prodrug and at least one pharmaceutically acceptable vehicle. A pharmaceutical composition can comprise a therapeutically effective amount of at least one propofol prodrug and at least one pharmaceutically acceptable vehicle. Pharmaceutically acceptable vehicles include diluents, adjuvants, excipients, and carriers. Pharmaceutical compositions may be produced using standard procedures (see, e.g., Remington's The Science and Practice of Pharmacy, 21st edition, Lippincott, Williams & Wilcox, 2005). Pharmaceutical compositions may take any form appropriate for oral delivery such as solutions, suspensions, emulsions, tablets, pills, pellets, granules, capsules, capsules containing liquids, powders, and the like. Pharmaceutical compositions provided by the present disclosure may be formulated so as to provide immediate, sustained, or delayed release of a propofol prodrug after administration to a patient by employing procedures known in the art (see, e.g., Allen et al., “Ansel's Pharmaceutical Dosage Forms and Drug Delivery Systems,” 8th edition, Lippincott, Williams & Wilkins, August 2004).

Pharmaceutical compositions may include an adjuvant that facilitates absorption of a propofol prodrug through the gastrointestinal epithelia. Such enhancers may, for example, open the tight-junctions in the gastrointestinal tract or modify the effect of cellular components, such as p-glycoprotein and the like. Suitable enhancers include alkali metal salts of salicylic acid, such as sodium salicylate, caprylic, or capric acid, such as sodium caprylate or sodium caprate, sodium deoxycholate, and the like. P-glycoprotein modulators are described in Fukazawa et al., U.S. Pat. No. 5,112,817 and Pfister et al., U.S. Pat. No. 5,643,909. Absorption enhancing compounds and materials are described in Burnside et al., U.S. Pat. No. 5,824,638, and Meezam et al., U.S. Application Publication No. 2006/0046962. Other adjuvants that enhance permeability of cellular membranes include resorcinol, surfactants, polyethylene glycol, and bile acids. Adjuvants may also reduce enzymatic degradation of a compound of a propofol prodrug. Microencapsulation using protenoid microspheres, liposomes, or polysaccharides may also be effective in reducing enzymatic degradation of administered compounds.

Propofol prodrugs provided by the present disclosure may be formulated in unit oral dosage forms. Unit oral dosage forms refer to physically discrete units suitable for dosing to a patient undergoing treatment, with each unit containing a predetermined quantity of a propofol prodrug. Oral dosage forms comprising at least one propofol prodrug may be administered to patients as a dose, with each dose comprising one or more oral dosage forms. A dose may be administered once a day, twice a day, or more than twice a day, such as three or four times per day. A dose may be administered at a single point in time or during a time interval. Oral dosage forms comprising at least one propofol prodrug may be administered alone or in combination with other drugs for treating the same or different disease, and may continue as long as required for effective treatment of the disease. Oral dosage forms comprising a propofol prodrug may provide a concentration of propofol in the plasma, blood, or tissue of a patient over time, following oral administration of the dosage form to the patient. The propofol concentration profile may exhibit an AUC that is proportional to the dose of the propofol prodrug.

A dose comprises an amount of a propofol prodrug calculated to produce an intended therapeutic effect. An appropriate amount of a propofol prodrug to produce an intended therapeutic effect will depend, in part, on the oral bioavailability of propofol provided by the propofol prodrug, by the pharmacokinetics of the propofol prodrug, and by the properties of the dosage form used to administer the propofol prodrug. A therapeutically effective dose of a propofol prodrug may comprise from about 10 mg-equivalents to about 5,000 mg-equivalents of propofol, from about 50 mg-equivalents to about 2,000 mg-equivalents of propofol, and in certain embodiments, from about 100 mg-equivalents to about 1,000 mg-equivalents of propofol. In certain embodiments, a therapeutically effective dose of a propofol prodrug provides a blood concentration of propofol from about 10 ng/mL to about 5,000 ng/mL, in certain embodiments from about 100 ng/mL to about 2,000 ng/mL, and in certain embodiments from about 200 ng/mL to about 1,000 ng/mL for a continuous period of time following oral administration of a dosage form comprising a propofol prodrug to a patient. In certain embodiments, a therapeutically effective dose of a propofol prodrug provides a blood concentration of propofol that is therapeutically effective for treating a disease in a patient, and that is less than a concentration effective to cause sedation in the patient, for example, less than about 1,500 ng/mL or less than about 2,000 ng/mL. In certain embodiments, a therapeutically effective dose of a propofol prodrug provides a blood concentration of propofol that is therapeutically effective and that is less than a concentration effective for the maintenance of general anesthesia (e.g., a sub-hypnotic concentration), for example, less than about 3,000 ng/mL or less than about 10,000 ng/mL.

Oral dosage forms comprising a propofol prodrug may have immediate release and/or controlled release characteristics. Immediate release oral dosage forms release the propofol prodrug from the dosage form within about 30 minutes following ingestion. In certain embodiments, an oral dosage form provided by the present disclosure may be a controlled release dosage form. Controlled delivery technologies may improve the absorption of a drug in a particular region or regions of the gastrointestinal tract. Controlled drug delivery systems may be designed to deliver a drug in such a way that the drug level is maintained within a therapeutically effective blood concentration range for a period as long as the system continues to deliver the drug at a particular rate. Controlled drug delivery may produce substantially constant blood levels of a drug as compared to fluctuations observed with immediate release dosage forms. For some diseases maintaining a controlled concentration of propofol in the blood or in a tissue throughout the course of therapy is desirable. Immediate release dosage forms may cause blood levels to peak above the level required to elicit the desired response, which may cause or exacerbate side effects. Controlled drug delivery may result in optimum therapy, reduce the frequency of dosing, and reduce the occurrence, frequency, and/or severity of side effects. Examples of controlled release dosage forms include dissolution controlled systems, diffusion controlled systems, ion exchange resins, osmotically controlled systems, erodable matrix systems, pH independent formulations, gastric retention systems, and the like.

The appropriate oral dosage form for a particular propofol prodrug may depend, at least in part, on the gastrointestinal absorption properties of the propofol prodrug, the stability of the propofol prodrug in the gastrointestinal tract, the pharmacokinetics of the propofol prodrug, and the intended therapeutic profile of propofol. An appropriate controlled release oral dosage form may be selected for a particular propofol prodrug. For example, gastric retention oral dosage forms may be appropriate for propofol prodrugs absorbed primarily from the upper gastrointestinal tract, and sustained release oral dosage forms may be appropriate for propofol prodrugs absorbed primarily from the lower gastrointestinal tract.

Gastric retention dosage forms, i.e., dosage forms designed to be retained in the stomach for a prolonged period of time, can increase the bioavailability of drugs that are most readily absorbed from the upper gastrointestinal tract. The residence time of a conventional dosage form in the stomach is 1 to 3 hours. After transiting the stomach, there is approximately a 3 to 5 hour window of bioavailability before the dosage form reaches the colon. However, if the dosage form is retained in the stomach, the drug can be released before it reaches the small intestine and will enter the intestine in solution in a state in which it can be more readily absorbed. Another use of gastric retention dosage forms is to improve the bioavailability of a drug that is unstable to the basic conditions of the intestine (see, e.g., Hwang et al., Critical Reviews in Therapeutic Drug Carrier Systems, 1998, 15, 243-284). To enhance drug absorption from the upper gastrointestinal tract, several gastric retention dosage forms have been developed. Examples include, hydrogels (see, e.g., Gutierrez-Rocca et al., U.S. Application Publication No. 2003/0008007), buoyant matrices (see, e.g., Lohray et al., Application Publication No. 2006/0013876), polymer sheets (see, e.g., Mohammad, Application Publication No. 2005/0249798), microcellular foams (see, e.g., Clarke et al., Application Publication No. 2005/0202090), and swellable dosage forms (see, e.g., Edgren et al., U.S. Application Publication No. 2005/0019409; Edgren et al., U.S. Pat. No. 6,797,283; Jacob et al., U.S. Application Publication No. 2006/0045865; Ayres, U.S. Application Publication No. 2004/0219186; Gusler et al., U.S. Pat. No. 6,723,340; Flashner-Barak et al., U.S. Pat. No. 6,476,006; Wong et al., U.S. Pat. Nos. 6,120,803 and 6,548,083; Shell et al., U.S. Pat. No. 6,635,280; and Conte et al., U.S. Pat. No. 5,780,057).

In swelling and expanding systems, dosage forms that swell and change density in relation to the surrounding gastric content may be retained in the stomach for longer than conventional dosage forms. Dosage forms can absorb water and swell to form a gelatinous outside surface and float on the surface of gastric content surface while maintaining integrity before releasing a drug. Fatty materials may be added to impede wetting and enhance flotation when hydration and swelling alone are insufficient. Materials that release gases may also be incorporated to reduce the density of a gastric retention dosage form. Swelling also may significantly increase the size of a dosage form and thereby impede discharge of the non-disintegrated swollen solid dosage form through the pylorus into the small intestine. Swellable dosage forms may be formed by encapsulating a core containing drug and a swelling agent, or by combining a drug, swelling agent, and one or more erodible polymers.

Gastric retention dosage forms may also be in the form of folded thin sheets containing a drug and water-insoluble diffusible polymer that opens in the stomach to its original size and shape so as to be sufficiently large to prevent or inhibit passage of the expanded dosage form through the pyloric sphincter.

Floating and buoyancy gastric retention dosage forms are designed to trap gases within sealed encapsulated cores that can float on the gastric contents, and thereby be retained in the stomach for a longer time, e.g., 9 to 12 hours. Due to the buoyancy effect, these systems provide a protective layer preventing the reflux of gastric content into the esophageal region and may also be used for controlled release devices. A floating system may, for example, contain hollow cores containing drug coated with a protective membrane. The trapped air in the cores floats the dosage form on the gastric content until the soluble ingredients are released and the system collapses. In other floating systems, cores comprise drug and chemical substances capable of generating gases when activated. For example, coated cores, comprising carbonate and/or bicarbonate generate carbon dioxide in the reaction with hydrochloric acid in the stomach or incorporated organic acid in the system. The gas generated by the reaction is retained to float the dosage form. The inflated dosage form later collapses and clears from the stomach when the generated gas permeates slowly through the protective coating.

Bioadhesive polymers may also provide vehicles for controlled delivery of drugs to a number of mucosal surfaces in addition to the gastric mucosa (see, e.g., Mathiowitz et al., U.S. Pat. No. 6,235,313; and Illum et al., U.S. Pat. No. 6,207,197). Bioadhesive systems can be designed by incorporation of a drug and other excipients within a bioadhesive polymer. On ingestion, the polymer hydrates and adheres to the mucus membrane of the gastrointestinal tract. Bioadhesive polymers may be selected that adhere to a desired region or regions of the gastrointestinal tract. Bioadhesive polymers may be selected to optimized delivery to targeted regions of the gastrointestinal tract including the stomach and small intestine. The mechanism of the adhesion is thought to be through the formation of electrostatic and hydrogen bonding at the polymer-mucus boundary. Jacob et al., U.S. Application Publication Nos. 2006/0045865 and 2005/0064027 disclose bioadhesive delivery systems useful for drug delivery to both the upper and lower gastrointestinal tract.

Ion exchange resins have also been shown to prolong gastric retention, potentially by adhesion.

Gastric retention oral dosage forms may be used for delivery of drugs that are absorbed mainly from the upper gastrointestinal tract. For example, certain propofol prodrugs may exhibit limited colonic absorption, and be absorbed primarily from the upper gastrointestinal tract. Thus, dosage forms that release a propofol prodrug in the upper gastrointestinal tract and/or retard transit of the dosage form through the upper gastrointestinal tract will tend to enhance the oral bioavailability of the propofol prodrug or propofol metabolite.

Polymer matrices have also been used to achieve controlled release of drug over a prolonged period of time. Sustained or controlled release may be achieved by limiting the rate by which the surrounding gastric fluid can diffuse through the matrix and reach the drug, dissolve the drug and diffuse out again with the dissolved drug, or by using a matrix that slowly erodes, continuously exposing fresh drug to the surrounding fluid. Disclosures of polymer matrices that function by these methods are found, for example, in Skinner, U.S. Pat. Nos. 6,210,710 and 6,217,903; Rencher et al., U.S. Pat. No. 5,451,409; Kim, U.S. Pat. No. 5,945,125; Kim, PCT International Publication No. WO 96/26718; Ayer et al., U.S. Pat. No. 4,915,952; Akhtar et al., U.S. Pat. No. 5,328,942; Fassihi et al., U.S. Pat. No. 5,783,212; Wong et al., U.S. Pat. No. 6,120,803; and Pillay et al., U.S. Pat. No. 6,090,411.

Other drug delivery devices that remain in the stomach for extended periods of time include, for example, hydrogel reservoirs containing particles (Edgren et al., U.S. Pat. No. 4,871,548); swellable hydroxypropylmethylcellulose polymers (Edgren et al., U.S. Pat. No. 4,871,548); planar bioerodible polymers (Caldwell et al., U.S. Pat. No. 4,767,627); polymers comprising a plurality of compressible retention arms (Curatolo et al., U.S. Pat. No. 5,443,843); hydrophilic water-swellable, cross-linked polymer particles (Shell, U.S. Pat. No. 5,007,790); and albumin-cross-linked polyvinylpyrrolidone hydrogels (Park et al., J. Controlled Release 1992, 19, 131-134).

In certain embodiments, propofol prodrugs may be practiced with a number of different dosage forms adapted to provide sustained release of a propofol prodrug upon oral administration. Sustained release oral dosage forms may be used to release drugs over a prolonged time period and are useful when it is desired that a drug or drug form be delivered to the lower gastrointestinal tract. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art (see, for example, “Remington's Pharmaceutical Sciences,” Lippincott, Williams & Wilkins, 21st edition, 2005, Chapters 46 and 47; Langer, Science 1990, 249, 1527-1533; and Rosoff, “Controlled Release of Drugs,” 1989, Chapter 2). Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art (see, for example, “Remington's: The Science and Practice of Pharmacy,” Lippincott, Williams & Wilkins, 21st edition, 2005, Chapters 46 and 47; Langer, Science 1990, 249, 1527-1533; and Rosoff, “Controlled Release of Drugs,” 1989, Chapter 2).

In diffusion-controlled systems, water-insoluble polymers control the flow of fluid and the subsequent egress of dissolved drug from the dosage form. Both diffusional and dissolution processes are involved in release of drug from the dosage form. In reservoir devices, a core comprising a drug is coated with the polymer, and in matrix systems, the drug is dispersed throughout the matrix. Cellulose polymers such as ethylcellulose or cellulose acetate can be used in reservoir devices. Examples of materials useful in matrix systems include methacrylates, acrylates, polyethylene, acrylic acid copolymers, polyvinylchloride, high molecular weight polyvinylalcohols, cellulose derivates, and fatty compounds such as fatty acids, glycerides, and carnauba wax.

In dissolution-controlled systems, the rate of dissolution of a drug is controlled by slowly soluble polymers or by microencapsulation. Once the coating is dissolved, the drug becomes available for dissolution. By varying the thickness and/or the composition of the coating or coatings, the rate of drug release can be controlled. In some dissolution-controlled systems, a fraction of the total dose may comprise an immediate-release component. Dissolution-controlled systems include encapsulated/reservoir dissolution systems and matrix dissolution systems. Encapsulated dissolution systems may be prepared by coating particles or granules of drug with slowly soluble polymers of different thickness or by microencapsulation. Examples of coating materials useful in dissolution-controlled systems include gelatin, carnauba wax, shellac, cellulose acetate phthalate, and cellulose acetate butyrate. Matrix dissolution devices may be prepared, for example, by compressing a drug with a slowly soluble polymer carrier into a tablet form.

The rate of release of drug from osmotic pump systems is determined by the inflow of fluid across a semipermeable membrane into a reservoir, which contains an osmotic agent. The drug is either mixed with the agent or is located in a reservoir. The dosage form contains one or more small orifices from which dissolved drug is pumped at a rate determined by the rate of entrance of water due to osmotic pressure. As osmotic pressure within the dosage form increases, the drug is released through the orifice(s). The rate of release is constant and may be controlled within limits yielding relatively constant blood concentrations of the drug. Osmotic pump systems may provide a constant release of drug independent of the environment of the gastrointestinal tract. The rate of drug release may be modified by altering the osmotic agent and the size of the one or more orifices.

Release of drug from erosion-controlled systems is determined by the erosion rate of a carrier polymer matrix. Drug is dispersed throughout the polymer matrix and the rate of drug release depends on the erosion rate of the polymer. The drug-containing polymer may degrade from the bulk and/or from the surface of the dosage form.

Sustained release oral dosage forms may be in any appropriate form suitable for oral administration, such as, for example, in the form of tablets, pills, or granules. Granules may be filled into capsules, compressed into tablets, or included in a liquid suspension. Sustained release oral dosage forms may additionally include an exterior coating to provide, for example, acid protection, ease of swallowing, flavor, identification, and the like.

Sustained release oral dosage forms may release a propofol prodrug from the dosage form to facilitate the ability of the propofol prodrug and/or propofol metabolite to be absorbed from an appropriate region of the gastrointestinal tract, for example, in the small intestine, or in the colon. In certain embodiments, sustained release oral dosage forms release a propofol prodrug from the dosage form over a period of at least about 4 hours, at least about 8 hours, at least about 12 hours, at least about 16 hours, at least about 20 hours, and in certain embodiments, at least about 24 hours. In certain embodiments, sustained release oral dosage forms release a propofol prodrug from the dosage form in a delivery pattern in which from about 0 wt % to about 20 wt % of the propofol prodrug is released in about 0 to about 4 hours, about 20 wt % to about 50 wt % of the propofol prodrug is released in about 0 to about 8 hours, about 55 wt % to about 85 wt % of the propofol prodrug is released in about 0 to about 14 hours, and about 80 wt % to about 100 wt % of the propofol prodrug is released in about 0 to about 24 hours. In certain embodiments, sustained release oral dosage forms release a propofol prodrug from the dosage form in a delivery pattern in which from about 0 wt % to about 20 wt % of the propofol prodrug is released in about 0 to about 4 hours, about 20 wt % to about 50 wt % of the propofol prodrug is released in about 0 to about 8 hours, about 55 wt % to about 85 wt % of the propofol prodrug is released in about 0 to about 14 hours, and about 80 wt % to about 100 wt % of the propofol prodrug is released in about 0 to about 20 hours. In certain embodiments, sustained release oral dosage forms release a propofol prodrug from the dosage form in a delivery pattern in which from about 0 wt % to about 20 wt % of the propofol prodrug is released in about 0 to about 2 hours, about 20 wt % to about 50 wt % of the propofol prodrug is released in about 0 to about 4 hours, about 55 wt % to about 85 wt % of the propofol prodrug is released in about 0 to about 7 hours, and about 80 wt % to about 100 wt % of the propofol prodrug is released in about 0 to about 8 hours.

Regardless of the specific form of oral dosage form used, a propofol prodrug may be released from the orally administered dosage form over a sufficient period of time to provide prolonged therapeutic concentrations of propofol in blood of a patient. Following oral administration, dosage forms comprising a propofol prodrug may provide a therapeutically effective concentration of propofol in the blood of a patient for a continuous time period of at least about 4 hours, of at least about 8 hours, for at least about 12 hours, for at least about 16 hours, and in certain embodiments, for at least about 20 hours following oral administration of the dosage form to the patient. The continuous period of time during which a therapeutically effective blood concentration of propofol is maintained may begin shortly after oral administration or following a time interval.

In certain embodiments, it may be desirable that the blood concentration of propofol be maintained at a level between a concentration that causes moderate sedation in the patient and a minimum therapeutically effective concentration for treating a disease such as alcohol withdrawal, central pain, anxiety, or pruritus for a continuous period of time. The blood concentration of propofol that causes moderate sedation (or anesthesia) in a patient can vary depending on the individual patient. Generally, a blood propofol concentration from about 1,500 ng/mL to about 2,000 ng/mL will produce moderate sedation, while a blood propofol concentration from about 3,000 ng/mL to about 10,000 ng/mL is sufficient to maintain general anesthesia. In certain embodiments, a minimum therapeutically effective blood propofol concentration will be about 10 ng/mL, about 20 ng/mL, about 50 ng/mL, about 100 ng/mL, about 100 ng/mL, about 200 ng/mL, about 400 ng/mL, or about 600 ng/mL. In certain embodiments, a therapeutically effective blood concentration of propofol for treating alcohol withdrawal, central pain, anxiety, or pruritus is from about 10 ng/mL to less than about 5,000 ng/mL, and in certain embodiments from about 10 ng/mL to less than about 2,000 ng/mL. In certain embodiments, a therapeutically effective blood concentration of propofol for treating alcohol withdrawal, central pain, anxiety, or pruritus is from about 10 ng/mL to less than a sedative concentration. In certain embodiments, a therapeutically effective blood concentration of propofol for treating alcohol withdrawal, central pain, anxiety, pruritus is from about 200 ng/mL to about 1,000 ng/mL. In certain embodiments, methods provided by the present disclosure provide a blood propofol concentration that, following oral administration to a patient, does not produce sedation and/or anesthesia in the patient.

A therapeutically effective propofol blood concentration for treating alcohol withdrawal, central pain, anxiety, or pruritus in a patient can also be defined in terms of the plasma concentration or pharmacokinetic profile. Thus, in certain embodiments, following oral administration of a dosage form comprising a propofol prodrug to a patient, the maximum propofol blood concentration, Cₘₐₓ, is less than the propofol blood concentration that causes sedation, for example, is less than about 1,500 ng/mL to about 2,000 ng/mL. In certain embodiments, following oral administration of a dosage form comprising a propofol prodrug to a patient, the propofol blood AUC during a 4-hour period may range from about 800 ng·h/mL to about 3,200 ng·h/mL and not cause sedation at any time following oral administration. In certain embodiments, following oral administration of a dosage form comprising a propofol prodrug to a patient, the propofol blood AUC during an 8-hour period may range from about 1,600 ng·h/mL to about 6,400 ng·h/mL and not cause sedation at any time following oral administration. In certain embodiments, following oral administration of a dosage form comprising a propofol prodrug to a patient, the propofol blood AUC during a 12-hour period may range from about 2,400 ng·h/mL to about 9,200 ng·h/mL and not cause sedation at any time following oral administration. In certain embodiments, following oral administration of a dosage form comprising a propofol prodrug to a patient, the propofol blood AUC during a 16-hour period may range from about 3,200 ng·h/mL to about 12,800 ng·h/mL and not cause sedation at any time following oral administration. In certain embodiments, following oral administration of a dosage form comprising a propofol prodrug to a patient, the propofol blood AUC during a 32-hour period may range from about 4,000 ng·h/mL to about 16,000 ng·h/mL and not cause sedation at any time following oral administration.

Propofol prodrugs may be absorbed from the gastrointestinal tract and enter the systemic circulation intact. In certain embodiments, a propofol prodrug exhibits an oral bioavailability of the propofol prodrug greater than about 40% that of an equivalent intravenous dose of the propofol prodrug, greater than about 60%, and in certain embodiments greater than about 80%. In certain of the foregoing embodiments, a propofol prodrug exhibits an oral bioavailability of propofol greater than about 10% that of an equivalent intravenous dose of propofol, greater than about 20%, greater than about 40%, and in certain embodiments greater than about 60%.

### Methods of Use

Propofol prodrugs that provide a high oral bioavailability of propofol and dosage forms comprising such propofol prodrug may be used to treat diseases such as alcohol withdrawal, central pain, anxiety, or pruritus. Methods provided by the present disclosure comprise treating alcohol withdrawal, central pain, anxiety, or pruritus in a patient by administering to a patient in need of such treatment a therapeutically effective amount of at least one propofol prodrug that provides a high oral bioavailability of propofol.

### Alcohol Withdrawal

### Delirium Tremens

Alcoholism or alcohol dependence is a chronic disorder with genetic, psychosocial, and environmental causes. About 16 million Americans are considered heavy drinkers (e.g., people who consume five or more alcoholic beverages per day during five or more days in the previous month), and 700,000 patients receive treatment for alcohol dependence each day (see Kenna, et al., Am. J. Health-Syst Pharm 2004, 61, 2272-9, 2381-8). Alcohol dependence accounts for approximately 100,000 deaths per year in the United States and results in direct and indirect social costs $185 billion annually with more than 70% of the cost attributed to lost productivity (10^{th} Special Report to the U.S. Congress on Alcohol and Health. Secretary of Health and Human Services. Rockville, Md.; U.S. Dept. of Health and Human Services, Public Health Service, National Institutes on Alcohol Abuse and Alcoholism, NIH Publication No. 00-1583, 1, June 2000). It is estimated that about 10% of Americans are affected by alcohol dependence at some point during their lifetime.

Alcohol withdrawal (291.81) and alcohol intoxication delirium/alcohol withdrawal delirium (291.0) are separately classified alcohol-induced disorders in DSM-IV (Diagnostic and Statistical Manual of Mental Disorders, Fourth Ed., Text Revision, Washington D.C., American Psychiatric Association, 2000; and see also, e.g., Bayard et al., Am Fam Physician 2004, 69, 1443-50). Alcohol withdrawal is the presence of a characteristic withdrawal syndrome that develops after the cessation of or reduction in heavy and prolonged alcohol use. A diagnosis of alcohol withdrawal is made when two or more symptoms including autonomic hyperactivity, e.g., sweating or pulse rate greater than 100, increased had tremor, insomnia, nausea or vomiting, transient visual, tactile, or auditory hallucinations or illusions, psychomotor agitation, anxiety, or grand mal seizures, develop within several hours to a few days after cessation or a reduction in alcohol use, and the symptoms cause clinically significant distress or impairment in social, occupational, or other important areas of functioning not due to a general medical condition or are better accounted for by another mental disorder. Alcohol withdrawal delirium, a symptom of alcohol withdrawal, includes disturbances in consciousness and cognition and visual, tactile or auditory hallucinations. Development of alcohol withdrawal delirium is often associated with a clinically relevant medical condition such as liver failure, pneumonia, gastrointestinal bleeding, sequelae of head trauma, hypoglycemia, an electrolyte imbalance, or postoperative status.

A delirium that occurs during substance intoxication is diagnosed as substance intoxication delirium, and a delirium that occurs during substance withdrawal is diagnosed as substance withdrawal delirium. Delirium that occurs during substance intoxication usually arises within minutes to hours after taking relatively high doses of certain drugs. Descriptive features of substance intoxication delirium include disturbance of consciousness, i.e., reduced clarity of awareness of the environment, with reduced ability to focus, sustain, or shift attention; a change in cognition such as memory deficit, disorientation, language disturbance, of the development of a perceptual disturbance that is not better accounted for by a preexisting, established, or evolving dementia; and the disturbance develops over a short period of time, usually hours to days, and tends to fluctuate during the course of the day. In addition, there must be evidence from the history, physical examination, or laboratory findings of substance intoxication or withdrawal, medication side effects, and/or toxin exposure considered to be etiologically related to the delirium. Other features associated with substance intoxication delirium include a disturbance in the sleep-wake cycle; disturbed psychomotor behavior such as restlessness or hyperactivity or alternatively sluggishness and lethargy, that may shift from one extreme to the other over the course of a day; emotional disturbances such as anxiety, fear, depression, irritability, anger, euphoria, and apathy; hallucinations or delusions; and non-specific neurological abnormalities such as tremor, myoclonus, asterixis, and reflex or muscle tone changes.

Alcohol intoxication delirium/alcohol withdrawal delirium is a subset of substance induced delirium. Delirium tremens is a delirium associated with alcohol withdrawal, i.e., alcohol withdrawal delirium. Delirium tremens usually develops from 2 to 10 days following alcohol withdrawal at which time the person is initially anxious and later develops increasing confusion, sleeplessness, nightmares, excessive sweating, and profound depression accompanied by an increased pulse rate and fever. Episodes may escalate to include hallucinations. As the delirium tremens progresses, the hands develop persistent tremor that sometimes extends to the head and body, and most people become severely uncoordinated. Delirium tremens can be fatal, particularly when untreated.

The use of alcohol affects many of the regulatory systems in the body, including an increase in the release of endogenous opiates, activation of the γ-aminobutyric acid A (GABA_{A}) receptor, inhibition of the N-methyl-D-aspartate (NMDA) receptor, and interactions with both serotonin and dopamine receptors. The GABA_{A} receptor us a ligand-gated chloride (Cl⁻) ion channel. When the GABA_{A} receptor is activated by the neurotransmitter GABA, the ion channel opens, allowing an influx of Cl⁻ ions through the postsynaptic membrane. This leads to an inhibitory effect via hyperpolarization of the nerve ending. When ethanol interacts with the GABA_{A} receptor, it augments the GABA activity on the receptor, thus enhancing the opening of the ion channel. This allows more Cl⁻ ions to flow into the nerve terminal. Thus, there is an augmented inhibitory effect without an increase in the affinity or amount of GABA binding to the receptor. During ethanol withdrawal, when there is no ethanol present, the influx of Cl⁻ ions decreases with the same amount of GABA binding to the receptor (Grobin et al., Psychopharmacology 1998, 139, 2-19). This results in decreased inhibitory effect and thus relative increase in nerve firing. The increase in neural activity may lead to some of the manifestations associated with delirium tremens, such as tremors and autonomic stimulation. The second receptor affected by ethanol is the NMDA receptor. Ethanol has been shown to inhibit the excitatory function of the NMDA receptor by the excitatory neurotransmitter glutamate (Tsai and Coyle, Annu Rev Med 1998, 49, 173-184). Patients with a history of chronic alcohol use have an up-regulation of these receptors in the CNS. During alcohol withdrawal, the inhibition is removed, allowing for an increase in excitatory conduction in the affected portion of the CNS. During alcohol withdrawal, the inhibition is removed, allowing for an increase in excitatory conduction in the affected portion of the CNS. This reaction is potentiated by the increased number of NMDA receptors during up-regulation. This mechanism may account for certain manifestations associated with delirium tremens such as increased heart rate, blood pressure, and tremors.

There is evidence that propofol can be effective in treating alcohol withdrawal, and more specifically delirium tremens (see, e.g., DeBellis et al., J. Intensive Care Med. 2005, 20(3), 164-73; McCowan and Marik, Crit Care Med 2000, 28(6), 1781-4; Coomes and Smith, Ann Emerg Med 1997, 30, 825-828; Takeshita, J Clin Psychiatry 2004, 65(1), 134-5; and Stiebel et al., Psychosomatics 1994, 35, 193. In these studies, propofol was effective in reducing the symptoms of delirium tremens at sedative and subsedative doses of propofol.

The mechanism of action of propofol is believed to be similar to the action of alcohol on the central nervous system. Propofol directly activates the GABA_{A} receptor chloride ionophore complex, increasing chloride conductance. In addition, propofol inhibits the NMDA subtype of glutamate receptor, possibly through an allosteric modulation of channel gating thereby depressing glutamate synaptic transmission. The action of propofol on the GABA_{A} and glutamate receptors may explain the efficacy in refractory status epilepticus and delirium tremens. Other sites of action believed to contribute to the pharmacological activity of propofol include sensitivity to glycine receptors (Hales and Lambert, Br. J Pharmacol 1991, 104, 619), inhibition of nicotinic receptor function (Violet et al., Anesthesiology 1997, 86, 760; and Furuya et al., Anesth Analg 1999, 88, 174), interactions with G-protein coupled receptors (Nagase et al., J Pharmacol 1999, 79, 319), and interactions with voltage-dependent sodium channels (Frenkel and Urban, Eur J Pharmacol 1991, 208, 75-79; and Ratnakumari and Hemmings, Br. J Pharmacol 1996, 119, 1498). Benzodiazepine is used to treat alcohol withdrawal and delerium tremens, and its activity is believed to be mediated via GABA_{A} receptors. Propofol has been shown to be effective in treating benzodiazepine refractory delirium tremens, suggesting that the effects of propofol are mediated by different receptors. (Coomes and Smith, Ann Emerg Med 1997, 30, 825-828; and McCowan and Marik, Crit Care Med 2000, 28(6), 1781-4).

The efficacy of compounds of Formulae (I)-(IV) and compositions thereof for treating alcohol withdrawal may be assessed using animal models and using clinical studies. Animal models of alcohol withdrawal and in particular alcohol withdrawal seizures using Withdrawal Seizure Prone mice are known (Beadles-Bohling and Wiren, Genes, Brain and Behavior 2006, 5, 483-496; Becker and Hale, Alcoholism: Clin Experimental Res 1993, 17(11), 94-98; and Crabbe, Eur J Pharmacology 1992, 221, 85-90). Clinical protocols for assessing the efficacy of propofol prodrugs of Formulae (I)-(IV) for treating alcoholism are described, for example in Scott et al., CNS Drugs 2005, 19(5), 445-464. The revised Clinical Institute Withdrawal Assessment for Alcohol (CIWA-Ar) scale can be used to quantify the severity of alcohol withdrawal syndrome (Sullivan et al., Br J Addict 1989, 84, 1353-70).

### Central Pain

Central pain syndromes represent a form of neuropathic pain that is associated with lesions of the brain or the spinal cord after a stroke or other traumatic injury (Cohen and Abdi, Curr Opin Anaesthesiol 2002, 15, 575-581; Canavero and Bonicalzi, Clin Neuropharmacol 2004, 27(4), 182-186; Nicholson, Am Acad Neurol 2004, 62, 530-536; and Devulder et al., Acta Neurol Belg 2002, 102, 97-103). For example, central pain can occur after lesions of the spinal cord caused by injury, syringomyelia, infarction, tumor, and myeletis, or cerebral lesions of nonvascular origin such as multiple sclerosis and tumor. Aberrant neural activity in deafferentiated circuits and a postlesion imbalance between facilitatory and inhibitory neural pathways are proposed to be features of central pain. Central pain manifests as burning pain, ice-like, tingling, shooting, tiring and bursting sensations. Characteristic symptoms of central pain include muscle pains, dysesthesias, hyperpathia, allodynia, shooting/lancinating pain, circulatory pain, and peristaltic/visceral pain.

Spinal cord injury pain (SCIP) and central post-stroke pain (CPSP) are central pain syndromes having differing etiologies. The predominant cause of SCI pain is trauma, however other causes include iatrogenic pain, inflammation, neoplasm, vascular disease, infection, and congenital disease (Siddal et al., Spinal Cord 1997, 35, 69-75; and Sjölund, Brain Res Rev 2002, 40, 250-256). CPSP is neuropathic pain following a cerebrovascular accident (Hansson, Eur J Neurology 2004, 11(Suppl), 22-30; and Widar et al., J Rehabil Med 2002, 34, 165-170). Central pain is estimated to occur in about 2% to about 8% of all stroke patients. Any lesion by infarction, bleeding or injury to the dorsal horn, ascending pathways of the spinal cord, brainstem, thalamus, subcortical white matter and cerebral cortex can induce neuropathic CPSP. The onset of central pain following a stroke occurs more than 1 month after the stroke in 40% to 60% of patients.

Glutamate receptors have been demonstrated to play a role in central sensitization. In humans, NMDA receptors have been implicated in central pain (Eide et al., Neurosurgery 1995, 37, 1080-1087). In a rodent model of central pain after spinal hemisection, the intrathecal administration of both NMDA, and non-NMDA AMPA/kainite receptor antagonists resulted in dose-dependent reductions in mechanical allodynia (Bennett et al., Brain Res 2000, 859, 72-82). In clinical studies, both NMDA glutamate receptor antagonist as such as ketamine (Backonja et al., Pain 1994, 56, 51-57; and Wood and Sloan, Palliative Med, 1997, 11, 57-58), and drugs that inhibit the secretion of glutamine from afferent neurons such as lamotrigine (Cavavero and Bonicalzi, Pain 1996, 68, 179-181; and Vestergaard et al., Neurology 2001, 56, 184-190) have been demonstrated to have beneficial effects on central pain.

Propofol, a NMDA receptor antagonist, has been shown to be effective in the treatment of central pain such as pain associated with trigeminal neuralgia (Kubota et al., Exp Brain Res. 2007, 179(2), 181-190), and SCIP and CPSP (Canavero et al., J Neurol 1995, 242(9), 561-7; and Canavero and Bonicalzi, Pain 1998, 74(2-3), 109-14). For example, subanesthetic doses of propofol that are ten times less (0.2 mg/kg IV) than a dose used to abolish migraine (100 mg IV) was effective in reducing both SCIP and CPSP central pain (Canavero and Bonicalzi, Clin Neuropharmacol 2004, 27(4), 182-186).

The efficacy of a propofol prodrug provided by the present disclosure for treating central pain can be assessed using a clinical trial (see e.g., Vestergaard et al., Neurology 2001, 56, 184-190; Attal et al., Neurology 2000, 54, 564-574; and Leijon and Boivie, Pain 1989, 36, 27-36), such as a randomized, crossover double-blind clinical trial, and/or using animal models of central pain (Xu et al., Pain 1992, 48(2), 279-90; Hao and Xu, Pain 1996, 66, 313-319; and Hao et al., Eur J Pharmacology 2006, 553, 135-140).

### Anxiety

Anxiety is defined and categorized in the Diagnostic and Statistical Manual of Mental Disorders, 4^{th} Ed., Text Revision (DSM-IV-TR), American Psychiatric Assoc., 2000, pages 429-484. Anxiety disorders include panic attack, agoraphobia, panic disorder without agoraphobia, agoraphobia without history of panic disorder, specific phobia, social phobia, obsessive-compulsive disorder, posttraumatic stress disorder, acute stress disorder, generalized anxiety disorder, anxiety disorder due to a general medical condition, substance-induced anxiety disorder, and anxiety disorder not otherwise specified.

Neurochemical investigations have linked anxiety to dysfunction in central GABAergic, serotonergic, and noradrenergc systems. Because of it role in neuronal function and its wide distribution in the brain, the anxiolytic effect of propofol may result from its GABA-potentiating action. The GABA_{A} receptor complex plays a major role in the pharmacology of anxiety and propofol potentiates GABA_{A} receptor-mediated effects. Propofol is thought to act on GABA neurons by facilitating the interaction of the inhibitory neurotransmitter GABA with its GABA_{A} receptor complex, enhancing the affinity of this receptor for GABA, and thereby facilitating the inhibitory actions of GABA. Inhibition of 5-HT receptor activity in the dorsal hippocampus has also been postulated as a mechanism underlying the anxiolytic action of propofol (Barann et al., Neuropharmacology 2000, 39(6), 1064-1074; and Matsuo et al., NeuroReport 1997, 8, 3087-3090). Other studies suggest that inhibition of NO synthesis and the consequent effects on the L-arginine activated NO-cGMP pathway may also be involved in the effect of propofol on anxiety (Volke et al., NeuroReport 1995, 6, 1285-1288).

Propofol has been shown to act as an anxiolytic, especially in the surgical setting (Rothermel, Curr Opin Pediatr 2003, 15(2), 200-3; and Bal et al., Eur J Anaesthesiol 2006, 23(6), 470-5). Animal studies demonstrate that propofol exerts an anxiolytic effect independent of any sedative effect (Pain et al., Anesthesiology 1999, 90(1), 191-196; and Kurt et al., Pol J Pharmacol 2003, 55, 973-77).

Useful animal models for assessing treatment of anxiety include fear-potentiated startle (Brown et al., J Experimental Psychol 1951, 41, 317-327); elevated plus-maze (Pellow et al., J Neurosci. Methods 1985, 14, 149-167; and Hogg, Pharmacol Biochem Behav 1996, 54(1), 21-20); fear-potentiated behavior in the elevated plus-maze test (Korte and De Boer, Eur J Pharmacol 2003, 463, 163-175); X-maze test of anxiety (Handley and Mithani, Arch Pharmacol 1984, 327, 1-5); and rat social interaction test (File, J Neurosci Methods 1980, 2, 219-238). Genetic animal models of anxiety are known (Toh, Eur J Pharmacol 2003, 463, 177-184) as are other animal models sensitive to anti-anxiety agents (Martin, Acta Psychiatr Scand 1998, Suppl 393, 74-80).

In clinical trials, efficacy can be evaluated using psychological procedures for inducing experimental anxiety applied to healthy volunteers and patients with anxiety disorders (see e.g., Graeff, et al., Brazilian J Medical Biological Res 2003, 36, 421-32) or by selecting patients based on the Structured Clinical interview for DSM-IV Axis I Disorders as described by First et al., Structured Clinical Interview for DSM-IV Axis I Disorders, Patient Edition (SCIDIP), Version 2. Biometrics Research, New York State Psychiatric Institute, New York, 1995. One or more scales can be used to evaluate anxiety and the efficacy of treatment including, for example, the Penn State Worry Questionnaire (Behar et al., J Behav Ther Exp Psychiatry 2003, 34, 25-43), the Hamilton Anxiety and Depression Scales, the Spielberger State-Trait Anxiety Inventory, and the Liebowitz Social Anxiety Scale (Hamilton, J Clin Psychiatry 1980, 41, 21-24; Spielberger and Vagg, J Personality Assess 1984, 48, 95-97; and Liebowitz, J Clin Psychiatry 1993, 51, 31-35 (Suppl)).

### Pruritus

Pruritis or itch is a common sensation that causes a person to want to scratch. Pruritis is a complex process that may negatively impact quality of life. Dermatological diseases and conditions associated with pruritus include urticaria; scabies; cutaneous T-cell lymphoma; dermatitis herpetiformis; pediculosis; pemphigoid; dermatophytosis; folliculitis; sunburn; lichen planus; atopic eczema; irritant contact dermatitis; allergic contact dermatitis; asteatoic dermatitis; mastocytosis; lichen simplex chronicus; psoriasis; miliaria; xerotic eczema. (see e.g., Moses, Am Fam Physician 2003, 68(6), 1135-1142). Systemic diseases and conditions associated with pruritis include cholestasis, chronic renal failure, delusions of parasitosis, Hodgkin's lymphoma, human immunodeficiency virus infection, hyperthyroidism, iron deficiency anemia, malignant carcinoid, multiple myeloma, neurodermatitis or neurotic excoriations, parasitic infections such as filariasis, schistosomiasis, onchoncerciasis and trichinosis, parvovirus B19 infection, peripheral neuropathy such as brachioradial pruritus, herpes zoster, and notalgia paresthetica, polycythemia rubra vera, scleoderma, urticaria; endocrine disorders such as throtoxicosis, myxedema, and diabetes mellitus; leukemia; uremia; multiple sclerosis; drug hypersensitivity; pregnancy; aging (senile pruritus); postmenopause; Sjogren's syndrome; carcinoid syndrome; dumping syndrome; and lymphomas; and weight loss in eating disorders (see e.g., Krajnik et al., J Pain Symptom Management 2001, 21(2), 151-168; and Moses, Am Fam Physician 2003, 68(6), 1135-1142).

Pruritis is typically diagnosed based on the patient's history and a physical examination. Testing of skin scrapings, skin biopsy, and/or skin culture can be useful for diagnosing skin-related pruritus if the dermatitis is not typical for a benign disease. Laboratory testing such as determination of thyroid-stimulating hormone levels, serum bilirubin and alkaline phosphatase levels, serum creatinine and blood urea nitrogen levels, complete blood count, and HIV and chest radiograph can also be used to evaluate the cause of the pruritis.

Propofol at subhypnotic doses has been shown to be effective in controlling pruritus associated with cholestasis and spinal morphine administration (Borgeat et al., Anesthesiology 1992, 76, 510-512; Borgeat et al., Am J Gastroenterol 1992, 87, 672-674; Borgeat et al., Gastroenterology 1993, 104, 244-247; Canavero, Med Hypotheses 1994, 42, 203-207; Bergasa, J Hepatology 2005, 43, 1078-1088; Horta et al., Br J Anaesthesia 2006, 96(6), 796-800; and Kostopanagiotou et al., Eur J Anaesthesiol 2006, 23(5), 418-21).

Useful animal models for assessing the efficacy of treating pruritus caused by psoriasis (Schön, J Invest Dermatology 1999, 112(4), 405-410), dermatitis (Shiohara et al., J Dermatological Sci 2004, 36, 1-9; Takano et al., Eur J Pharmacology 2004, 495, 159-165; Umeda et al., Life Sciences 2006, 79, 2144-2150; and Marsella and Olivry, Clinics Dermatology 2003, 21, 122-133; Nojima and Carstens, J Neurosci Methods 2003, 126, 137-143); cholestasis (Inan and Cowan, Pharmacol Biochem Behav 2006, 85, 39-43); and autoimmune disease (Umeuchi et al., Eur J Pharmacol 2005, 518, 133-139), are known.

### Dose

The amount of a propofol prodrug that will be effective in the treatment of a particular disease, disorder, or condition disclosed herein will depend on the nature of the disease, disorder, or condition, and can be determined by standard clinical techniques known in the art. In addition, in vitro or in vivo assays may optionally be employed to help identify optimal dosage ranges. The amount of a compound administered can depend on, among other factors, the patient being treated, the weight of the patient, the health of the patient, the disease being treated, the severity of the affliction, the route of administration, the potency of the compound, and the judgment of the prescribing physician.

For systemic administration, a therapeutically effective dose may be estimated initially from in vitro assays. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in humans. One having ordinary skill in the art may optimize administration to humans based on animal data.

In certain embodiments, a therapeutically effective dose of a propofol prodrug may comprise from about 1 mg-equivalents to about 2,000 mg-equivalents of propofol per day, from about 5 mg-equivalents to about 1000 mg-equivalents of propofol per day, and in certain embodiments, from about 10 mg-equivalents to about 500 mg-equivalents of propofol per day.

A dose may be administered in a single dosage form or in multiple dosage forms. When multiple dosage forms are used the amount of a propofol prodrug contained within each of the multiple dosage forms may be the same or different.

In certain embodiments, an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, a pharmaceutical composition may exhibit a high therapeutic index. The data obtained from these cell culture assays and animal studies may be used in formulating a dosage range that is not toxic for use in humans. A dose of a highly orally bioavailable propofol prodrug maybe within a range of circulating concentrations in for example the blood, plasma, or central nervous system, that is therapeutically effective, that is less than a sedative dose, and that exhibits little or no toxicity. A dose may vary within this range depending upon the dosage form employed.

During treatment a dose and dosing schedule may provide sufficient or steady state systemic concentrations of a therapeutically effective amount of propofol to treat a disease. In certain embodiments, an escalating dose may be administered.

Propofol prodrugs that provide a high oral bioavailability of propofol may be administered orally, and may be administered at intervals for as long as necessary to obtain an intended or desired therapeutic effect.

### Combination Therapy

Propofol prodrugs that provide a high oral bioavailability propofol may be used in combination therapy with at least one other therapeutic agent. Propofol prodrugs and another therapeutic agent(s) can act additively or, and in certain embodiments, synergistically. In some embodiments, propofol prodrugs may be administered concurrently with the administration of another therapeutic agent, such as for example, a compound for treating alcohol withdrawal, central pain, anxiety, or pruritus. In some embodiments, propofol prodrug may be administered prior or subsequent to administration of another therapeutic agent, such as for example, a compound for treating alcohol withdrawal, central pain, anxiety, or pruritus.

Methods provided by the present disclosure include administering one or more propofol prodrugs and one or more other therapeutic agents provided that the combined administration does not inhibit the therapeutic efficacy of the one or more propofol prodrugs and/or other therapeutic agent and/or does not produce adverse combination effects.

In certain embodiments, propofol prodrugs may be administered concurrently with the administration of another therapeutic agent, which may be part of the same pharmaceutical composition or dosage form as or in a different composition or dosage form than that containing a propofol prodrug. When a propofol prodrug is administered concurrently with another therapeutic agent that potentially can produce adverse side effects including, but not limited to, toxicity, the therapeutic agent may be administered at a dose that falls below the threshold at which the adverse side effect is elicited.

In certain embodiments, propofol prodrugs may be administered prior or subsequent to administration of another therapeutic agent. In certain embodiments of combination therapy, the combination therapy comprises alternating between administering a propofol prodrug and a composition comprising another therapeutic agent, e.g., to minimize adverse side effects associated with a particular drug.

In certain embodiments, propofol prodrugs provided by the present disclosure may be administered to a patient together with one or more compounds useful for treating alcohol withdrawal and/or delirium tremens. Useful compounds for treating alcohol withdrawal and/or delirium tremens include benzodiazepines such as alprazolam, bromazepam, chlordiazepoxide, clobazam, clonazepam, clorazepate, diazepam, estazolam, flurazepam, halazepam, ketazolam, lorazepam, nitrazepam, oxazepam, prazepam, quazepam, temazepam, and traizolam; beta-adrenergic blocking agents such as acebutolol, atenolol, betazolol, bisoprolol, carteolol, labetalol, metoprolol, madolol, oxprenolol, penbutlol, pindolol, propranolol, sotalol, and timolol; barbiturates such as amobarbital, aprobarbital, butabarbital, mephobarbital, metharbital, pentobarbital, phenobarbital, and secobarbital; sedative-hypnotic agents such as chlormethiazole, flunitrazepam, pentobarbital, paraldehyde, barbital, and midazolam; neuroleptic agents such as haloperidol, and atenolol; antipsychotics such as chlorpromazine, promazine, perphenazine, and chlordiazepoxide; beta-adrenergic antagonists; magnesium; thiamine; carbamazepine; dexamethasone; physostigmine; 5-hydroxytryptophan; bromperidol; clonidine; phenyloin; baclofen; gabapentin; pregabalin; and vigabatrin.

Other examples of drugs useful for treating alcohol dependency or alcohol abuse disorders include disulfuram, naltrexone, acamprosate, ondansetron, atenolol, chlordiazepoxide, clonidine, clorazepate, diazepam, oxazepam, methadone, topiramate, 1-alpha-acetylmethadol, buprenorphine, bupropion, and baclofen.

In certain embodiments, propofol prodrugs provided by the present disclosure may be administered to a patient together with one or more compounds useful for treating central pain. Compounds useful for treating central pain include tricyclic antidepressants such as amitriptyline; antiepileptic drugs such as lamotrigine, gabapentin, and baclofen; ketamine; midazolam; lidocaine; clonidine; and opioids such as morphine.

In certain embodiments, propofol prodrugs provided by the present disclosure may be administered to a patient together with one or more compounds useful for treating anxiety disorders. Examples of useful compounds for treating anxiety disorders include anxiety disorders include alprazolam, atenolol, busipirone, chlordiazepoxide, clonidine, clorazepate, diazepam, doxepin, escitalopram, halazepam, hydroxyzine, lorazepam, nadolol, oxazepam, paroxetine, prochlorperazine, trifluoperazine, venlafaxine, amitriptyline, sertraline, citalopram, clomipramine, fluoxetine, fluvoxamine, and paroxetine.

In certain embodiments, propofol prodrugs provided by the present disclosure may be administered to a patient together with one or more compounds or therapies useful for treating pruritis. Examples of compounds useful for treating pruritus include antihistamines such as azatadine, brompheniramine, cetirizine, chlorpheniramine, clemastine, cyproheptadine, desloratadine, desloratadine, dexchlorpheniramine, imenhydrinate; diphenhydramine, doxylamine, fexofenadine, hydroxyzine, loratadine, phenindamine, doxepin, and cetirizine; cannabinoid receptor agonists such as dronabinol; anesthetics such as benzocaine, diperodon, lidocaine, and pramoxine; selective serotonin reuptake inhibitors such as fluvoxamine, fluoxetine, and paroxetine; corticosteroids such as prednisone; antidepressants such as mirtazapine; calcineurin inhibitors such as pimecrolimus and tacrolimus; opiate receptor antagonists such as naltrexone; rifampicin; benzocaine; hydrocortisone; lidocaine; ondansetron; thalidomide; carbamazepine; gabapentin; capsaicin; cholestyramine; ursodiaol acid; pimozide; aspirin; interferon alfpha; and nalbuphine. UV light therapy, cutaneous field stimulation, herbal remedies, nutritional remedies, reflex therapy, acupuncture, and hydrotherapy have also been shown to be effective to some extent in treating pruritis.

### EXAMPLES

The following examples describe in detail methods of using propofol prodrugs. It will be apparent to those skilled in the art that many modifications, both to materials and methods, may be practiced without departing from the scope of the disclosure.

### Example 1

### Pharmacokinetics of Compound (2) and Propofol in Rats

Propofol or compound (2) was administered as an intravenous bolus injection or by oral gavage to groups of four to six adult male Sprague-Dawley rats (about 250 g). Animals were conscious at the time of the experiment. When orally administered, propofol or compound (2) was administered as an aqueous solution at a dose equivalent to propofol per kg body weight. When administered intravenously, propofol was administered as a solution (Diprivan®, Astra-Zeneca) at a dose equivalent to 10 or 15 mg of propofol per kg body weight. Animals were fasted overnight before the study and for 4 hours post-dosing. Blood samples (0.3 mL) were obtained via a jugular vein cannula at intervals over 8 hours following oral dosing. Blood was quenched immediately using acetonitrile with 1% formic acid and then was frozen at −80° C. until analyzed.

Three hundred (300) μL of 0.1% formic acid in acetonitrile was added to blank 1.5 mL tubes. Rat blood (300 μL) was collected at different times into tubes containing EDTA and vortexed to mix. A fixed volume of blood (100 μL) was immediately added into the Eppendorf tube and vortexed to mix. Ten (10) μL of a propofol standard stock solution (0.04, 0.2, 1, 5, 25, and 100 μg/mL) was added to 90 μL of blank rat blood quenched with 300 μL of 0.1% formic acid in acetonitrile. Then, 20 μL of p-chlorophenylalanine was added to each tube to make a final calibration standard (0.004, 0.02, 0.1, 0.5, 2.5, and 10 μg/mL). Samples were vortexed and centrifuged at 14,000 rpm for 10 min. The supernatant was analyzed by LC/MS/MS.

An API 4000 LC/MS/MS spectrometer equipped with Agilent 1100 binary pumps and a CTC HTS-PAL autosampler and a Phenomenex Synergihydro-RP 4.6×30 mm column were used in the analysis. The mobile phase for propofol analysis was (A) 2 mM ammonium acetate, and (B) 5 mM ammonium acetate in 95% acetonitrile. The mobile phase for the analysis of compound (2) was (A) 0.1% formic acid, and (B) 0.1% formic acid in acetonitrile. The gradient condition was: 10% B for 0.5 min, then to 95% B in 2.5 min, then maintained at 95% B for 1.5 min. The mobile phase was returned to 10% B for 2 min. An APCI source was used on the API 4000. The analysis was done in negative ion mode for propofol and in positive ion mode for compound (2). The MRM transition for each analyte was optimized using standard solutions. Five (5) μL of each sample was injected. Non-compartmental analysis was performed using WinNonlin (v.3.1 Professional Version, Pharsight Corporation, Mountain View, Calif.) on individual animal profiles. Summary statistics on major parameter estimates was performed for Cₘₐₓ (peak observed concentration following dosing), Tₘₐₓ (time to maximum concentration is the time at which the peak concentration was observed), AUC₀₋ₜ T (area under the serum concentration-time curve from time zero to last collection time, estimated using the log-linear trapezoidal method), AUC_{(0-∞)}, (area under the serum concentration time curve from time zero to infinity, estimated using the log-linear trapezoidal method to the last collection time with extrapolation to infinity), and t_{1/2} (terminal half-life).

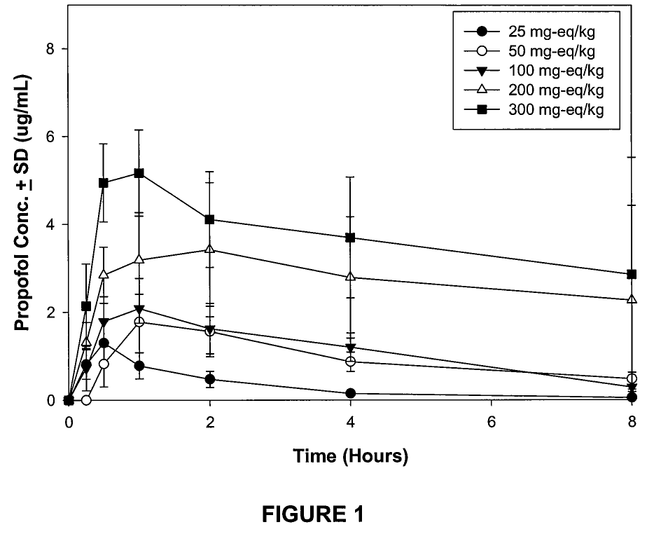

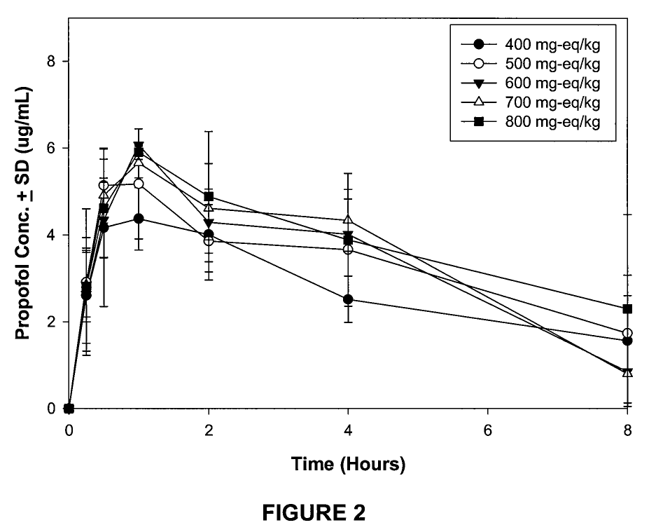

The oral bioavailability (F %) of propofol was determined by comparing the area under the propofol concentration vs time curve (AUC) following oral administration of compound (2) with the AUC of the propofol concentration vs time curve following intravenous administration of propofol on a dose normalized basis. The results from these studies are summarized in FIG. 1 (at doses of 25 mg-eq/kg, 50 mg-eq/kg, 100 mg-eq/kg, 200 mg-eq/kg, and 300 mg-eq/kg propofol), FIG. 2 (at doses of 400 mg-eq/kg, 500 mg-eq/kg, 600 mg-eq/kg, 700 mg-eq/kg, and 800 mg-eq/kg propofol), and Table 1.

TABLE 1
Pharmacokinetic Parameter Summary for Rat Study

| Compound | | | | | | |
|---|---|---|---|---|---|---|
| (2) | | | | | | |
| Dose | | | | | | |
| (mg-eq/kg | Cₘₐₓ | Tₘₐₓ | T_{1/2-1} | AUCₜ | AUC_{inf} | Fₚₒ |
| propofol) | (μg/mL) | (hr) | (hr) | (hr · μg/mL) | (hr · μg/mL) | (%) |
| 25 | 0.8 (0.2) | 1.7 (0.5) | 2.2 (0.5) | 2.5 (0.7) | 2.8 (0.7) | 65 (17) |
| 50 | 2.0 (0.8) | 2.0 (1.2) | 3.2 (2.9) | 5.0 (1.7) | 6.0 (2.0) | 78 (23) |
| 100 | 2.2 (0.4) | 1.1 (0.6) | 2.8 (0.7) | 9.1 (1.3) | 10.3 (0.9) | 61 (6) |
| 200 | 3.4 (2.0) | 1.3 (1.1) | 5.0 (4.3) | 18.5 (12.2) | 24.6 (6.7) | 72 (20) |
| 300 | 4.6 (0.7) | 0.8 (0.4) | 2.4 (0.4) | 18.7 (0.5) | 20.9 (0.1) | 41 (0) |
| 400 | 4.7 (0.7) | 1.0 (0.7) | 2.6 (0.8) | 22.0 (2.7) | 25.0 (1.2) | 37 (2) |
| 500 | 5.0 (0.6) | 2.3 (1.7) | 11.1 (0.1) | 41.7 (17.5) | 53.4 (23.9) | 83 (28) |
| 600 | 6.1 (0.0) | 1.0 (0.0) | 2.4 (0.0) | 25.4 (22.4) | 33.4 (11.2) | 33 (11) |
| 700 | 5.6 (0.3) | 1.0 (0.0) | 3.8 (2.7) | 24.3 (5.2) | 40.1 (18.7) | 39 (21) |
| 800 | 6.0 (0.5) | 1.3 (0.6) | 6.1 (5.7) | 29.5 (11.9) | 60.6 (53.6) | 60 (53) |

### Example 2

### Pharmacokinetics of Compound (2) and Propofol in Dogs

Compound (2) or propofol was administered by oral gavage or as an intravenous bolus injection, respectively, to groups of two to four adult male Beagle dogs (about 8 kg) as solutions in water. Animals were fasted overnight before the study and for 4 hours post-dosing. Blood samples (1.0 mL) were obtained via the femoral vein at intervals over 24 hours after oral dosing. Blood was quenched immediately using acetonitrile with 1% formic acid and then frozen at −80° C. until analyzed. Compound (2) was administered to dogs with a minimum of 7-day wash out period between dosing sessions.

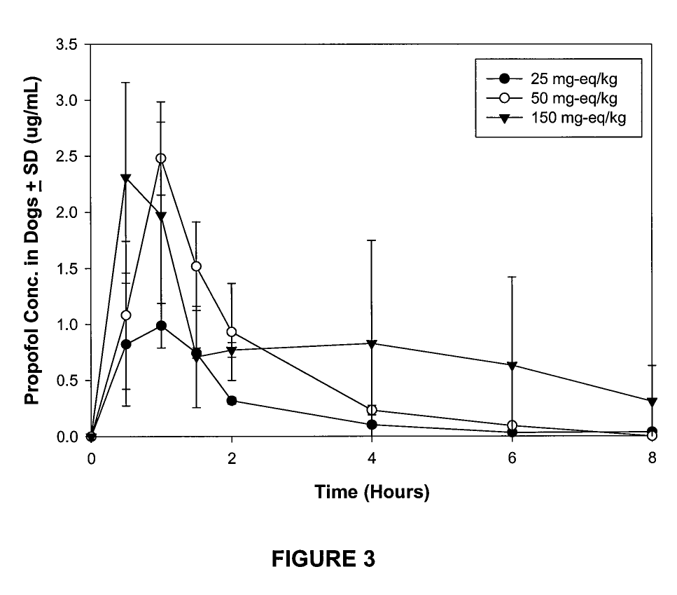

Blood sample preparation and LC/MS/MS analysis were the same as for the rat study described in Example 1. The pharmacokinetics of propofol following oral administration of compound (2) at doses of 25 mg-eq/kg, 50 mg-eq/kg, and 150 mg-eq/kg propofol to dogs is summarized in FIG. 3 and Table 2.

TABLE 2
Pharmacokinetic Parameter Summary for Dog Study

| Compound | | | | | | |
|---|---|---|---|---|---|---|
| (2) | | | | | | |
| Dose Level | | | | | | |
| (mg-eq/kg | Cₘₐₓ | Tₘₐₓ | T_{1/2-1} | AUCₜ | AUC_{inf} | Fₚₒ |
| propofol) | (μg/mL) | (hr) | (hr) | (hr · μg/mL) | (hr · μg/mL) | (%) |
| 25 | 1.0 (0.3) | 0.8 (0.4) | 0.9 (0.1) | 1.8 (0.5) | 2.0 (0.5) | 37 (10) |
| 50 | 2.5 (0.3) | 1.0 (0.0) | 1.1 (0.1) | 4.3 (0.7) | 4.4 (0.7) | 41 (6) |
| 150 | 2.3 (0.8) | 0.5 (0.0) | 2.3 (0.6) | 6.7 (5.0) | 7.9 (6.5) | 25 (20) |

### Example 3

### Toxicity Studies

Acute toxicity studies in rats were undertaken to assess the tolerance of a single oral dose of compound (2) formulated in water. The results indicated that compound (2) was well tolerated at levels from about 49 mg-eq/kg to about 1552 mg-eq/kg propofol of administered compound. Transient hypoactivity was observed at doses from about 49 mg-eq/kg up to about 388 mg-eq/kg propofol within about 30 minutes of dose and maintained up to 4 hours post dose. Sedation was observed at doses from about 582 mg-eq/kg up to about 970 mg-eq/kg propofol within about 1.5 hours of dosing and continued up to 4 hours post dose. Anesthesia was observed at doses from about 1164 mg-eq/kg up to about 1552 mg-eq/kg propofol within about 1 hour of dosing and continued up to about 2 hours post dose. Complete recovery from hypoactivity, sedation, and anesthesia occurred in all rats within about 8 hours after dosing. Doses above about 1552 mg-eq/kg (about 800 mg-eq/kg of propofol) were not tested.

Acute toxicity studies were also performed by orally administering a single dose of compound (2) formulated in water to groups of male beagle dogs at doses from about 25 mg-eq/kg to about 150 mg-eq/kg propofol. Results indicated that at these doses compound (2) was well tolerated in dogs. No sedation or anesthesia was observed at these doses.

Multiple dose studies in rats were performed by orally administering compound (2) formulated in water to groups of male rats at doses of 49 mg-eq/kg to 97 mg-eq/kg propofol for a period of five days, by oral gavages administered once a day. No adverse effects were observed in the multiple dose studies. Results indicated that compound (2) was well tolerated by rats. No sedation or anesthesia was observed at these doses.

### Example 4

### Use of Animal Models to Assess the Efficacy of Compounds for Treating Alcohol Withdrawal

Withdrawal Seizure-Prone (WSP) and Withdrawal Seizure-Resistant (WSR) mice are used. Mice are made dependent on ethanol via 72 h of chronic ethanol vapor inhalation as described by Beadles-Bohling et al., Neurochem Int 2000, 37, 463-472. On day 1, mice are weighted, injected with a loading dose of ethanol and pyrazole HCl (Pyr), an alcohol dehydrogenase inhibitor, and placed into ethanol vapor chambers. Controls are placed into air chambers and receive pyrazole only. At 24 and 48 h, pyrazole boosters are administered to both the experimental and control groups. Blood ethanol concentrations (BECs) for ethanol groups are measured and the ethanol vapor concentrations adjusted to equate ethanol exposure between lines. Mean BECs are maintained between approximately 1.0-2.0 mg/mL, depending upon the effects of the test compound being studied. After 72 h, all mice are removed from the chambers to initiate withdrawal, and ethanol treated mice have blood samples drawn for BEC determination. Ethanol concentration can be determined by gas chromatography using the method described by Gallaher et al., J Pharmacol Exp Ther 1996, 277, 604-612.

Following removal from the ethanol or air chambers, mice are scored hourly for handling-induced convulsion (HIC) (Crabbe and Kosobud, Behav Genet. 1985, 15, 521-536; and Crabbe et al., J Pharamacol Exp Ther 1991, 257, 663-667). Scoring can begin 1 h after removal from ethanol and hourly over the next 12-15 h and again at 24 h. If animals do not return to baseline HIC levels by 25 h, an additional score is obtained at 48 h. The scale described by Crabbe et al can be used (0=no convulsion after a gently 180° spin; 1=only facial grimace after gentle 180° spin; 2=tonic convulsion elicited by gently 180° spin; 3=tonic-clonic convulsion after 180° spin; 4=tonic convulsion when lifted by tail, no spin; 4=tonic-clonic convulsion when lifted by tail, no spin; 6=severe tonic-clonic convulsion when lifted by tail, no spin; and 7=severe tonic-clonic convulsion elicited before lifting by the tail). The area under the curve is calculated and used to quantitatively evaluate withdrawal severity. An additional index of withdrawal severity is the peak HIC score, calculated by identifying the highest HIC for each individual mouse and averging this score with the two adjacent scores. Data are analyzed by appropriate statistical methdods.

### Example 5

### Animal Model for the Assessment of Central Pain Associated with Spinal Cord or Trigeminal Nerve Injury

Ischemic spinal cord injury is produced in female rats according to the methods described by Xu et al., Pain 1992, 48, 279-290. Rats are anesthetized and a midline incision is made on the skin overlying vertebral segments T12-L1. The animals are positioned beneath an argon laser beam and irradiated fro 10 min with the beam directed towards vertebral segment T12 or T13 (spinal segments L3-5). Immediately prior to and 5 min after the start of the irradiation, erythrosine B dissolved in 0.9% saline is injected intravenously through the tail vein at a dose of 32.5 mg/kg. A tunable argon ion laser operating at 514 nm is used. The average beam output power was 160 mW. The beam covers the entire width of the vertebra and the length of the beam is 1-2 mm. After irradiation, the wound is closed in layers and the rats are allowed to recover. The bladder is emptied manually for 1 week. The experiments are conducted in rats that have been injured 4-6 months earlier and they exhibit allodynia-like behavior for at least 3 months.

Mechanical and cold sensitivity after spinal cord injury are assessed using the following procedure. The vocalization threshold to graded mechanical touch/pressure stimuli are tested with von Frey hairs applied to the lower back and flank area. During testing the rats are gently restrained in a standing position and the von Frey hair is pushed onto the skin until the filament becomes bent. The frequency of stimulation is about 1/s and at each intensity the stimulus is applied 5-10 times. The intensity of stimulation that induces consistent vocalization (>75% response rate) is considered as the pain threshold.

The response of rats to bushing stimulation is tested with the blunt point of a pencil gently stroking the skin on the trunk in a rostro-caudal direction. The frequency of the stimulation is about 1/s and responses are graded with a score of 0=no observable response; 1=transient vocalization and moderate effort to avoid probe; 2=consistent vocalization and aversive reactions; and 3=sustained and prolonged vocalization and aggressive behaviors. Normal rats exhibit no reactions to brush stimuli (score 0).

Response to cold is tested with ethyl chloride spray applied to the shaved allodynic skin area located on the lower back and/or flanks. The response is graded.

Motor performance is evaluated using a combined motor testing system measuring open field locomotion, toe spread, righting reflex, extension withdrawal, placing reflex and inclined plane performance.

Measurements are taken 30 min, 1 h, 2 h, and 3 h after the acute administration of vehicle or test compound. A group of animals is also treated twice daily for 7 days. Measurements are taken immediately before and 1 h after the morning administration until 7 days after cessation of treatment.

A method described by Hao et al., Eur J Pharmacology 2006, 553, 135-140 and Vos et al., J Neurosci 1994, 2708-2723, involving photochemically-induced ischemic infraorbital nerve (IoN) injury can be used as a model of trigeminal pain. Male or female rats are anesthetized and the left IoN is exposed via a longitudinal incision at the maxillary region and all branches of the nerve are carefully lifted on a glass hook. A piece of aluminum foil is placed under the nerve and the nerve is irradiated for 6 min with a tunable argon ion laser as for spinal cord injury. The rat is positioned so that the laser beam is perpendicular and transversal to the exposed nerve. Immediately before irradiation erythrosine B is injected intravenously and the injection is repeated after 5 min. After irradiation the wound is closed in layers.

Mechanical sensitivity is tested with a series of von Frey filaments. Rats are gently held by an experimenter and the von Frey filaments are applied in ascending order to the IoN territory on the hairy skin of the vibrissal pad. The stimulation with each filament consisted of four consecutive applications at 1/s on the injured and than on the contralateral side. The response threshold is taken as the force at which the rat either exhibits a withdrawal reaction or escape/attack in 75% of trials. The rats are habituated to the testing procedure for several days before nerve injury and baseline response is determined in two sessions. Testing is done 3, 7, 10, and 14 d after the injury to assess the development of mechanical hypersensitivity. The effect of test compound is tested 14-16 days after injury when mechanical hypersensitivity is well established. Measurements are taken 30 min, 1 h, 2 h, and 3 h following drug injection.

### Example 6

### Elevated Plus-Maze Test for Assessment of Anxiety

A method for assessing the effects of compounds on anxiety described by Pellow and File, Pharmacol Biochem Behav 1986, 24, 524-529 is used. A plus-maze is consists of two open arms (50×10 cm) and two closed arms (50×10×40 cm). The arms extend from a central platform (10×10 cm) and are raised 50 cm. Each mouse is placed at the center of the maze facing a closed arm and is allowed to explore the maze for 5 min. The time spent in the open arms and the time spent in the closed arms is monitored, and the percent of time spent in the open arms determined. Increased time spent in the open arms indicates an anxiolytic effect for the test condition. A test that measures spontaneous locomotor activity such as measurement in an activity cage can be used to determine whether the test compound also affects locomotor activity. It is desirable that a compound exhibiting an anxiolytic effect not decrease locomotor activity.

### Example 7

### Assessment of Pruritus Associated with Cholestasis

A model described by Inan and Cowan, Pharmacol Biochem Behav 2006, 85, 39-43, that measures scratching behavior secondary to cholestasis induced by chronic α-ethynylestradiol injection in rats can be used to assess the efficacy of compounds. Male Sprague-Dawley rats weighing 175-200 g are used. The animals are housed 2 per cage with free access to food and water. A standard light-dark cycle is maintained.

Rats are injected subcutaneously (right or left flank areas) once a day with either vehicle (50% propylene glycol in distilled water) or 17 α-ethynylestradiol (EE) (2 mg/kg) (dissolved in 50% propylene glycol in distilled water) for 13 consecutive days. On day 13, before the daily injections, the animals are acclimated in individual rectangular observation boxes for at least 1 h. Immediately after the regular injection of either vehicle or EE, the number of body scratching movements with hind legs is counted for 30 min. The next day, only EE-injected rats are observed. After acclimation, these rats are divided into two groups and pretreated subcutaneously with either saline or test compound 20 min before EE and scratching is counted again for 30 min. Multiple doses of test compound may be used. At the end of 30 min observation, heart blood (4-5 mL) is drawn from both vehicle and EE-injected rats, the animals being anesthetized during the procedure. Blood is centrifuged to obtain serum. Serum bile acid, dynophin A, and NO levels are measured to confirm cholestasis. Data are analyzed using an appropriate statistical method.

### Example 8

### Assessment of Pruritus Associated with Atopic Dermatitis

A genetic animal model described by Takano et al., Eur J Pharmacol 2003, 471, 223-228, can be used to assess the antipruritic effect of compounds in humans with atopic dermatitis. Male NC/Nga mice are housed in a controlled environment. To increase the incidence of dermatitis-related scratching behavior, the mice are maintained with mice of the same strain having severe skin lesions and then used at 15-20 weeks of age.

The spontaneous scratching behavior of NC/Nga mice is measured as follows. Scratching is measured for 24 h after vehicle administration and before treatment with a test compound. The next day, the test compound is administered, and scratching behavior is measured for 24 h. For the measures, a small magnet (1 mm dia×3 mm long) is implanted subcutaneously into both the hind paws of each mouse under ether anesthesia at least 6 h before the scratching measurement. Each mouse is placed in an observation chamber (11 cm dia×18 cm high), which is surrounded by a round coil. The electric current induced in the coil by the movement of magnets attached to the hind paws is amplified and recorded. Measurements are also made following administration of vehicle (control) and compounds known to have antipruritic effects in this model such as dexamethasone and tacrolimus (Takano et al., Eur J Pharm 2004, 495, 159-165). The measured scratching behavior is statistically analyzed.

Finally, it should be noted that there are alternative ways of implementing the disclosures contained herein. Accordingly, the present embodiments are to be considered as illustrative and not restrictive, and the claims are not to be limited to the details given herein, but may be modified within the scope and equivalents thereof.

What is claimed is:

1. A method of treating a disease in a patient comprising orally administering to a patient in need of such treatment a therapeutically effective amount of a propofol prodrug having a high oral bioavailability, wherein the disease is chosen from alcohol withdrawal, central pain, anxiety, and pruritis.

2. The method of claim 1, wherein the propofol prodrug is a compound of Formula (I): 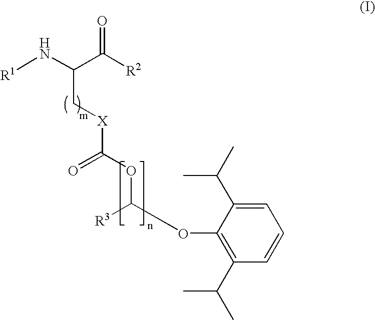
or a pharmaceutically acceptable salt thereof, wherein:
X is chosen from a bond, —CH₂—, —NR¹¹—, —O—, and —S—;
m is chosen from 1 and 2;
n is chosen from 0 and 1;
R¹ is chosen from hydrogen, [R⁵NH(CHR⁴)ₚC(O)]—, R⁶—, R⁶C(O)—, and R⁶OC(O)—;
R² is chosen from —OR⁷ and —[NR⁸(CHR⁹)_{q}C(O)OR⁷];
p and q are independently chosen from 1 and 2;
each R³ is independently chosen from hydrogen, alkyl, substituted alkyl, alkoxycarbonyl, aryl, substituted aryl, arylalkyl, carbamoyl, substituted carbamoyl, cycloalkyl, substituted cycloalkyl, heterocycloalkyl, heteroaryl, substituted heteroaryl, and heteroarylalkyl;
each R⁴ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, or when R⁴ and R⁵ are attached to adjacent atoms then R⁴ and R⁵ together with the atoms to which they are bonded form a ring chosen from a heterocycloalkyl and substituted heterocycloalkyl ring;
R⁵ is chosen from hydrogen, R⁶—, R⁶C(O)—, and R⁶OC(O)—;
R⁶ is chosen from alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;
R⁷ is chosen from hydrogen, alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;
R⁸ is chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl;
each R⁹ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, or when R⁸ and R⁹ are attached to adjacent atoms then R⁸ and R⁹ together with the atoms to which they are bonded form a ring chosen from a heterocycloalkyl and substituted heterocycloalkyl ring; and
R¹¹ is chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl;
with the provisos that:
when R¹ is [R⁵NH(CHR⁴)ₚC(O)]— then R² is —OR⁷; and
when R² is —[NR⁸(CHR⁹)_{q}C(O)OR⁷] then R¹ is not [R⁵NH(CHR⁴)ₚC(O)]—.

3. The method of claim 2, wherein the propofol prodrug is (S)-2-amino-3-(2,6-diisopropylphenoxycarbonyloxy)-propanoic acid, or a pharmaceutically acceptable salt thereof.

4. The method of claim 1, wherein the propofol prodrug is a compound of Formula (II): 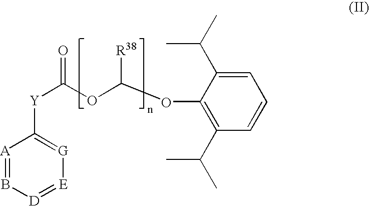
or a pharmaceutically acceptable salt thereof, wherein:
n is chosen from 0 and 1;
Y is chosen from a bond, CR²¹R²², NR²³, O, and S;
A is chosen from CR²⁴ and N;
B is chosen from CR²⁵ and N;
D is chosen from CR²⁶ and N;
E is chosen from CR²⁷ and N;
G is chosen from CR²⁸ and N;
R³⁸ is chosen from hydrogen, alkyl, substituted alkyl, alkoxycarbonyl, aryl, substituted aryl, arylalkyl, carbamoyl, substituted carbamoyl, cycloalkyl, substituted cycloalkyl, heterocycloalkyl, heteroaryl, substituted heteroaryl, and heteroarylalkyl;
R²¹ and R²² are independently chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl;
R²³ is chosen from hydrogen, alkyl, substituted alkyl, aryl, arylalkyl, cycloalkyl, and heteroaryl;
R²⁴ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;
R²⁵ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;
R²⁶ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;
R²⁷ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;
R²⁸ is chosen from hydrogen, alkyl, substituted alkyl, alkoxy, substituted alkoxy, alkoxycarbonyl, aryl, substituted aryl, arylalkyl, carboxyl, cycloalkyl, substituted cycloalkyl, heterocycloalkyl, halogen, heteroaryl, substituted heteroaryl, heteroarylalkyl, hydroxyl, and —W[C(O)]ₖZ(CR²⁹R³⁰)ᵣCO₂R³¹;
W is chosen from a bond, —CR³²R³³, —NR³⁴, O, and S;
Z is chosen from —CR³⁵R³⁶, —NR³⁷, O, and S;
k is chosen from 0 and 1;
r is chosen from 1, 2, and 3;
each of R²⁹, R³⁰, R³¹, R³², R³³, R³⁵, and R³⁶ is independently chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl; and
R³⁴ and R³⁷ are independently chosen from hydrogen, alkyl, substituted alkyl, aryl, arylalkyl, cycloalkyl, and heteroaryl;
with the provisos that:
at least one of A, B, D, E, and G is not N;
one and only one of R²⁴, R²⁵, R²⁶, R²⁷, or R²⁸ is —W[C(O)]ₖZ(CR²⁹R³⁰)ᵣCO₂R³¹; and
if k is 0 then W is a bond.

5. The method of claim 1, wherein the propofol prodrug is a compound of Formula (III): 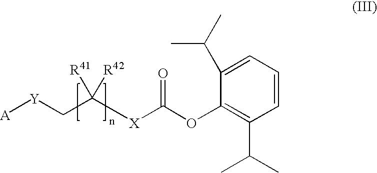
or a pharmaceutically acceptable salt thereof, wherein:
each R⁴¹ and R⁴² is independently chosen from hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl, or R⁴¹ and R⁴² together with the carbon atom to which they are bonded form a ring chosen from a cycloalkyl, substituted cycloalkyl, heterocycloalkyl, and substituted heterocycloalkyl ring;
A is chosen from hydrogen, acyl, substituted acyl, alkyl, substituted alkyl, aryl, substituted aryl, arylalkyl, substituted arylalkyl, heteroalkyl, substituted heteroalkyl, heteroaryl, substituted heteroaryl, heteroarylalkyl, and substituted heteroarylalkyl; or A, Y, and one of R⁴¹ and R⁴² together with the atoms to which they are bonded form a ring chosen from a heterocycloalkyl and substituted heterocycloalkyl ring;
Y is chosen from —O— and —NR⁴³—;
R⁴³ is chosen from hydrogen, alkyl, substituted alkyl, arylalkyl, and substituted arylalkyl;
n is an integer from 1 to 5;
X is chosen from —NR⁴⁴, —O—, —CH₂, and —S—; and
R⁴⁴ is chosen from hydrogen, alkyl, substituted alkyl, arylalkyl, and substituted arylalkyl.

6. The method of claim 1, wherein the propofol prodrug is a compound of Formula (IV): 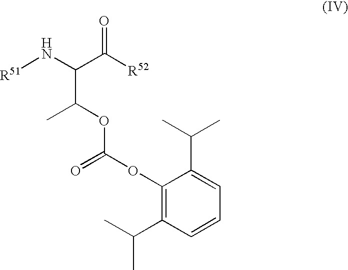
or a pharmaceutically acceptable salt thereof, wherein:
R⁵¹ is chosen from hydrogen, [R⁵⁵NH(CHR⁵⁴)ₚC(O)]—, R⁵⁶, R⁵⁶C(O)—, and R⁵⁶OC(O—;
R⁵² is chosen from —OR⁵⁷ and —[NR⁵⁸(CHR⁵⁹)_{q}C(O)OR⁵⁷];
p and q are independently chosen from 1 and 2;
each R⁵⁴ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, or when R⁵⁴ and R⁵⁵ are bonded to adjacent atoms then R⁵⁴ and R⁵⁵ together with the atoms to which they are bonded form a ring chosen from a heterocycloalkyl and substituted heterocycloalkyl ring;
R⁵⁵ is chosen from hydrogen, R⁵⁶—, R⁵⁶C(O)—, and R⁵⁶OC(O)—;
R⁵⁶ is chosen from alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;
R⁵⁷ is chosen from hydrogen, alkyl, substituted alkyl, aryl, substituted aryl, arylalkyl, substituted arylalkyl, cycloalkyl, substituted cycloalkyl, heterocycloalkyl, heteroaryl, substituted heteroaryl, and heteroarylalkyl;
R⁵⁸ is chosen from hydrogen, alkyl, substituted alkyl, aryl, substituted aryl, arylalkyl, cycloalkyl, substituted cycloalkyl, heterocycloalkyl, heteroaryl, substituted heteroaryl, and heteroarylalkyl; and
each R⁵⁹ is independently chosen from hydrogen, alkyl, substituted alkyl, alkoxy, substituted alkoxy, acyl, substituted acyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl, or when R⁵⁸ and R⁵⁹ are bonded to adjacent atoms then R⁵⁸ and R⁵⁹ together with the atoms to which they are bonded form a ring chosen from a heterocycloalkyl and substituted heterocycloalkyl ring;
with the proviso that when R⁵² is —[NR⁵⁸(CHR⁵⁹)_{q}C(O)OR⁵⁷] then R⁵¹ is not [R⁵⁵NH(CHR⁵⁴)ₚC(O)]—.

7. The method of claim 6, wherein the propofol prodrug is 2-amino-3-methyl-3-(2,6-diisopropyl-phenoxycarbonyloxy)-propanoic acid, or a pharmaceutically acceptable salt thereof.

8. The method of claim 1, comprising maintaining a propofol concentration in the blood of the patient from about 10 ng/mL to about 2,000 ng/mL for at least about 4 hours following oral administration of the propofol prodrug to the patient.

9. The method of claim 1, wherein the therapeutically effective amount is less than an amount that causes moderate sedation in the patient.

10. The method of claim 1, wherein the disease is alcohol withdrawal.

11. The method of claim 10, wherein the disease is delirium tremens.

12. The method of claim 1, wherein the disease is central pain.

13. The method of claim 1, wherein the disease is anxiety.

14. The method of claim 1, wherein the disease is pruritus.
